(19) United States
(12) Patent Application Publication
(10) Pub. No.: US 2020/0197682 A1
(43) Pub. Date: Jun. 25, 2020

(54) ACCESS DEVICE WITH GUIDEWIRE AND RELATED METHODS

(71) Applicant: Prytime Medical Devices, Inc., Boeme, TX (US)

(72) Inventors: Curtis J. Franklin, Lakewood, CO (US); Luke William Fisher, Lakewood, CO (US)

(21) Appl. No.: 16/386,503

(22) PCT Filed: Oct. 17, 2017
(86) PCT No.: PCT/US2017/056860

§ 371 Date: Apr. 17, 2019

Related U.S. Application Data
(60) Provisional application No. 62/456,390, filed on Feb. 8, 2017.
(60) Provisional application No. 62/415,744, filed on Nov. 1, 2016.
(60) Provisional application No. 62/409,000, filed on Oct. 17, 2016.

(51) Int. Cl. A61M 39/02; A61M 25/06; A61M 25/09; A61M 39/04; A61M 25/00; A61M 5/315

(52) U.S. Cl. CPC A61M 39/0247; A61M 25/0693; A61M 25/09; A61M 39/045; A61M 2039/0291; A61M 5/31511; A61M 2025/0003; A61M 2039/0258; A61M 2039/027; A61M 25/0097

(57) ABSTRACT

An access device for accessing a lumen of a blood vessel includes a hub, a needle having a tip and a guidewire having a rear end portion and a front end portion. The hub has a longitudinal axis, a main channel, a guidewire channel and an auxiliary channel. The needle has a proximal end and a needle lumen extending between the tip and the proximal end. The proximal end is secured to a distal end of the hub. The needle lumen is in fluid communication with the main channel. The guidewire has a rear end portion and a front end portion. The front end portion is positioned in the guidewire channel proximally relative to a sealing mechanism in an initial configuration. The sealing mechanism substantially limits fluid flow from the main channel through the guidewire channel.

### CROSS-REFERENCE TO RELATED APPLICATIONS

The present application claims the benefit of U.S. Provisional Patent Application No. 62/456,390, filed on Feb. 8, 2017 and titled, “Access Device with Guidewire and Related Method;” U.S. Provisional Patent Application No. 62/415,744, filed on Nov. 1, 2016 and titled, “Access Device with Guidewire and Related Method” and U.S. Provisional Patent Application No. 62/409,000, filed Oct. 17, 2016 and titled, “Access Device with Guidewire and Related Method,” the entire contents of which are incorporated herein by reference in their entirety.

### BACKGROUND OF THE INVENTION

Known arterial and vascular access procedures are conducted using a cannula needle and guidewire as distinct and separate components. Vascular access procedures may also utilize a separate syringe that is attached to a connecting portion to draw a vacuum on the needle and withdraw blood from the vein when the needle tip is positioned within the vein. The tip of the needle is inserted through the patient's skin and into the blood vessel (vein or artery), which may be difficult to locate and pierce due to differing patient anatomy. In addition, the proper placement of the needle in the blood vessel may be difficult to confirm due to limited pressure in the blood vessels (particularly in veins), positioning of different blood vessels near each other, patient anatomy and other factors. Appropriately positioning the needle tip in the vein may be facilitated by pulling a vacuum in the needle using a separately attached syringe to draw blood from the vessel, typically a vein, once the tip is positioned in the vessel. This vacuum is typically not required when accessing an artery, as arterial pressure typically causes blood to flow into and out of a proximal end of the needle when the tip is in the artery. When the tip is in the vessel, blood flow visually confirms the appropriate positioning of the needle. Proper positioning may, however, be lost while the medical professional reaches for and attempts to insert a guidewire into the needle or access device. The needle or access device may move such that the tip moves out of the targeted vessel because the patient or needle moves, the medical professional conducts an follow-up step related to the procedure, the medical professional moves the needle or access device while arranging the guidewire or for various other reasons.

Once the tip of the needle is positioned in the blood vessel, the physician or medical technician typically inserts a separate guidewire through the needle and into the blood vessel. The guidewire is typically retrieved from a nearby surgical table or other proximate area. The physician's or medical technician's movement to retrieve the guidewire can result in movement of the tip of the needle, potentially resulting in the tip moving out of the blood vessel. This movement in reaching or otherwise retrieving the guidewire also slows the procedure, which can be important in trauma situations and for patient comfort. Feeding the guidewire into the proximal end of the needle or connector following retrieval can also be difficult, resulting in additional potential for movement of the tip out of the blood vessel and delay in the procedure. For example, the physician may need to re-start the procedure of positioning the tip of the needle in the vessel if the tip moves out of the vessel or may need to re-arrange the tip back into the appropriate vessel, thereby delaying the procedure and often increasing patient discomfort. Feeding the guidewire into the proximal end of the needle is particularly difficult when the needle has a small diameter at its proximal end or the medical professional is rushing the procedure, particularly in trauma situations.

Traditional vascular access procedures may be performed in the field using a cannula needle and guidewire as individual components. For some access procedures, typically venous access, a needle is used with a syringe attached to the needle via a needle hub or Luer connection. The needle is inserted into the vessel while drawing back on the syringe plunger to create vacuum so that blood is drawn into the syringe upon access to the vein or as an indication of when the tip of the needle is positioned within the vein (presence of blood indicates the needle is in the vessel). Next, the syringe is removed from the needle, a guidewire is aligned with and inserted into a proximal end of the needle and the guidewire is advanced through the needle into the vessel. The guidewire is urged into the needle such that the guidewire is positioned within the vessel, preferably the vein, as a guide for subsequent instruments. The needle is then removed proximally over the guidewire with a front portion of the guidewire remaining in the vessel and a rear portion of the guidewire extending out of the patient's skin. The guidewire provides a path for insertion of other devices into the vessel and the other devices are typically used for treatment that is associated with the vessel. This multiple step process and exchange of components, including the needle, syringe, other instrument and guidewire may cause pain for the user and provides multiple opportunities for the needle or guidewire to move or become misaligned, potentially requiring the medical professional to re-start the procedure or realign the components, particularly the tip of the needle before the guidewire is inserted into the vessel.

The preferred access device with a guidewire addresses the above-described shortcomings of prior art blood vessel access devices and methods. For example, it would be desirable to design, develop and deploy an access device that eliminates the requirement to feed the guidewire into the needle after the tip of the needle is inserted into the patient's vessel to expedite the vessel access procedure. It would also be desirable to design, develop and deploy an access device that is quickly able to assess whether the tip of the access device is positioned in the patient's vessel without significant flash blood flow out of the access device that provides an indication of whether the tip is in an artery or a vein.

### BRIEF SUMMARY OF THE INVENTION

Briefly stated, the preferred invention is directed to an access device for accessing a lumen of a blood vessel and includes a hub, a needle having a tip and a guidewire having a rear end portion and a front end portion. The hub has a longitudinal axis, a main channel, a guidewire channel and an auxiliary channel. The needle has a proximal end and a needle lumen extending between the tip and the proximal end. The proximal end is secured to a distal end of the hub. The needle lumen is in fluid communication with the main channel. The front end portion of the guidewire is positioned in the guidewire channel proximally relative to a sealing mechanism in an initial configuration. The sealing mechanism substantially limits fluid flow from the main channel through the guidewire channel.

In another aspect, the preferred invention is directed to an access device for accessing a lumen of a blood vessel, wherein the access device includes a hub, a needle and a guidewire. The hub has a longitudinal axis, a main channel, a guidewire channel, a guidewire holder and an auxiliary channel. The main channel and the guidewire channel extend substantially parallel to the longitudinal axis. The auxiliary channel extends at an acute angle relative to the longitudinal axis. The needle has a tip, a proximal end and a needle lumen extending between the tip and the proximal end. The proximal end is secured to a distal end of the hub and the needle lumen is in fluid communication with the main channel. The needle lumen extends substantially parallel to the longitudinal axis. The guidewire has a rear end portion and a front end portion. The front end portion of the guidewire is positioned in the guidewire channel in an initial configuration and the rear end portion is positioned in the guidewire holder in the initial configuration. The front end portion of the guidewire is positioned proximally relative to a sealing mechanism in the guidewire channel. The sealing mechanism is positioned proximate the main channel substantially limit fluid flow from the main channel through the guidewire channel.

In a further aspect, the preferred present invention is directed to an access device for accessing a lumen of a blood vessel. The access device includes a hub, a needle and a pre-loaded guidewire. The hub has a longitudinal axis, a main channel, a guidewire channel, and an auxiliary channel that can be used to observe blood flow and/or monitor pressure. The needle has a tip and a proximal end. The proximal end is fixed to a distal end of the hub. The needle is in fluid communication with the main channel. The pre-loaded guidewire is positioned in the guidewire channel in an initial configuration and may extend into a main channel of the hub and a lumen of the needle in the initial configuration.

A preferred embodiment of the access device is designed to incorporate, secure or pre-load the guidewire into a body of the device and, alternatively, a syringe or mechanism to draw a vacuum on the needle. The preferred configuration of the access device allows the procedure of inserting the guidewire into the patient's vessel to be conducted faster, easier, and with greater repeatability. Most patient's need for intravenous (“IV”) fluid and drugs can be met with a percutaneous peripheral venous catheter. A cutdown procedure can be used to access the vessel when percutaneous catheter insertion is not feasible. Typical cutdown sites are the cephalic vein in the arm and the saphenous vein at the ankle. Cutdown is, however, rarely needed because of the recent popularity of peripherally inserted central catheter (“PICC”) lines and intraosseous lines in both adults and children. The PICC line is typically inserted into a relatively large vessel, preferably a vein, in a patient's arm, leg or neck such that antibiotics, nutrition, medications or other medicament can be quickly carried to the patient's heart.

In another aspect, the preferred invention is directed to an access device for accessing a lumen of a blood vessel. The preferred access device includes a hub, a needle and a guidewire. The hub includes a longitudinal axis, a main channel, a guidewire channel and an auxiliary channel. The needle has a tip, a proximal end and a needle lumen extending between the tip and the proximal end. The proximal end is secured or fixed to a distal end of the hub. The needle lumen is in fluid communication with the main channel. The guidewire has a rear end portion and a front end portion. The front end portion of the guidewire is positioned in or pre-loaded into the guidewire channel in an initial configuration. Pre-loading the guidewire into the guidewire channel eliminates the need for the medical professional to feed the separate guidewire into a proximal end of the needle after the tip of the needle is positioned in the appropriate vessel. Pre-loading allows the medical professional to quickly start feeding the guidewire into the vessel as soon as the tip is positioned in the vessel. The tip is preferably a J-tip, curved wire, straight wire or other shape that may have a shape or configuration that limits trauma to the vessel when inserted into the vessel. For example, the tip may have a J-shape or curved shape that uncoils upon exiting from the tip of the needle within the vessel.

In a further aspect, the preferred invention is directed to an access device for accessing a lumen of a blood vessel including a hub, a first needle fixed or secured to the distal end of the hub, a second needle fixed or secured to the distal end and a third needle fixed or secured to the distal end. The hub has a longitudinal axis, a proximal end and the distal end. The first, second and third needles extend substantially parallel to the longitudinal axis. The first, second and third needles define a needle plane and are positioned on the needle plane. The access device also includes a movable guidewire holder attached to the proximal end and a guidewire. The guidewire has a rear end portion and a front end portion. The front end portion is pre-loaded on the guidewire holder for selective alignment with the first, second or third needles.

Common complications (e.g., local infection, venous thrombosis, thrombophlebitis, interstitial fluid extravasation) of PICC lines, intraosseous lines, catheters and the like can be reduced by using a meticulous sterile technique during insertion, by conducting quick, consistent insertion procedures and by replacing or removing the catheters within seventy-two hours (72 hrs.).

Arterial catheters (also called intra-arterial catheters or A-lines) are common in critically ill patients and in trauma situations. The arterial catheters can also be used to obtain arterial blood for laboratory testing, for direct measurement of blood pressure and cardiac output and for related procedures. Insertion of an arterial catheter is an invasive procedure and complications can occur.

The preferred access device and procedure provides a platform for quick and consistent insertion of a guidewire into an arterial lumen for placement of a catheter or other procedure.

### BRIEF DESCRIPTION OF THE SEVERAL VIEWS OF THE DRAWINGS

The foregoing summary, as well as the following detailed description of preferred embodiments of the access device, instrument, implant and method of the preferred embodiments of the present invention, will be better understood when read in conjunction with the appended drawings. For the purposes of illustrating the access device with a pre-loaded guidewire and related methods, there are shown in the drawings preferred embodiments. It should be understood, however, that the preferred present invention is not limited to the precise arrangements and instrumentalities shown. In the drawings:

FIG. 1 is a side elevational view of an access device with a guidewire in accordance with a first preferred embodiment of the present invention;

FIG. 1A is a front perspective view of the access device of FIG. 1;

FIG. 1B is a cross-sectional view of the access device with guide wire of FIG. 1, taken along line 1B-1B of FIG. 1A;

FIG. 2 is a side elevational view of an access device in accordance with a second preferred embodiment of the present invention;

FIG. 2A is an alternative side elevational view of the access device of FIG. 2;

FIG. 2B is cross-sectional view of the access device of FIG. 2, taken along line 2B-2B of FIG. 2A;

FIG. 3 is a side elevational view of an access device in accordance with a third preferred embodiment of the present invention;

FIG. 3A is a top perspective view of the access device of FIG. 3;

FIG. 3B is a cross-sectional view of the access device of FIG. 3, taken along line 3B-3B of FIG. 3A;

FIG. 4 is a side elevational view of an access device in accordance with a fourth preferred embodiment of the present invention;

FIG. 4A is a top perspective view of the access device of FIG. 4, wherein the guidewire is not shown for clarity;

FIG. 4B is a cross-sectional view of the access device of FIG. 4, taken along line 4B-4B of FIG. 4A;

FIG. 5 is a side elevational, cross-sectional view of an access device in accordance with a fifth preferred embodiment of the present invention;

FIG. 6A is a side elevational view of an access device in accordance with a sixth preferred embodiment of the present invention;

FIG. 6B is a top perspective view of the access device of FIG. 6A;

FIG. 6C is a side perspective view of the access device of FIG. 6A;

FIG. 6D is a cross-sectional view of the access device of FIG. 6A, taken along line 6D-6D of FIG. 6C;

FIG. 7A is a top perspective view of an access device in accordance with a seventh preferred embodiment of the present invention;

FIG. 7B is a top plan view of the access device of FIG. 7A;

FIG. 7C is a rear perspective view of the access device of FIG. 7A;

FIG. 8A is a rear perspective view of an access device in accordance with an eighth preferred embodiment of the present invention;

FIG. 8B is a cross-sectional view of the access device of FIG. 8A, taken along line 8B-8B of FIG. 8A;

FIG. 9A is a side perspective view of an access device in accordance with a ninth preferred embodiment of the present invention;

FIG. 9B is a cross-sectional view of the access device of FIG. 9A, taken along line 9B-9B of FIG. 9A;

FIG. 9C is an alternative side perspective view of the access device of FIG. 9A, wherein an alternative pressure monitor is mounted to an attachment of the access device;

FIG. 9D is a side perspective view of the access device with guide wire of FIG. 9A, with a further alternative pressure monitor mounted to an attachment of the access device;

FIG. 9E is cross-sectional view of the access device of FIG. 9A, taken along line 9E-9E of FIG. 9D;

FIG. 9F is a magnified cross-sectional view of a distal portion of the access device of

FIG. 9A, wherein a guidewire is loaded into a needle lumen of the needle of the access device of FIG. 9A;

FIG. 9G is a front perspective view of the access device of FIG. 9A with an alternative needle connected to a hub of the device of FIG. 9A;

FIG. 9H is a magnified cross-sectional view of a distal portion of the access device of

FIG. 9A, wherein the alternative needle of FIG. 9G is connected to the hub;

FIG. 10A is a top perspective view of an access device in accordance with a tenth preferred embodiment of the present invention;

FIG. 10B is a magnified top plan view of the access device of FIG. 10A;

FIG. 10C is a cross-sectional view of the access device of FIG. 10A, taken along line 10C-10C of FIG. 10A;

FIG. 11A is a side perspective view of an access device in accordance with an eleventh preferred embodiment of the present invention;

FIG. 11B is a cross-sectional view of the access device of FIG. 11A, taken along line 11B-11B of FIG. 11A;

FIG. 11C is a magnified, side perspective, cross-sectional view of the access device of FIG. 11A, taken from within the circle 11C of FIG. 11B; and

FIG. 12 is a side perspective view of a sealing mechanism that may be utilized with any of the preferred access devices described herein.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one”. The words “right”, “left”, “lower” and “upper” designate directions in the drawings to which reference is made. The words “inwardly” or “distally” and “outwardly” or “proximally” refer to directions toward and away from, respectively, the patient's body, or the geometric center of the preferred access device with a guidewire and related parts thereof. The words, “anterior”, “posterior”, “superior,” “inferior”, “lateral” and related words and/or phrases designate preferred positions, directions and/or orientations in the human body or to directions and/or orientations of the access device and related parts to the patient's body to which reference is made and are not meant to be limiting. The terminology includes the above-listed words, derivatives thereof and words of similar import.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

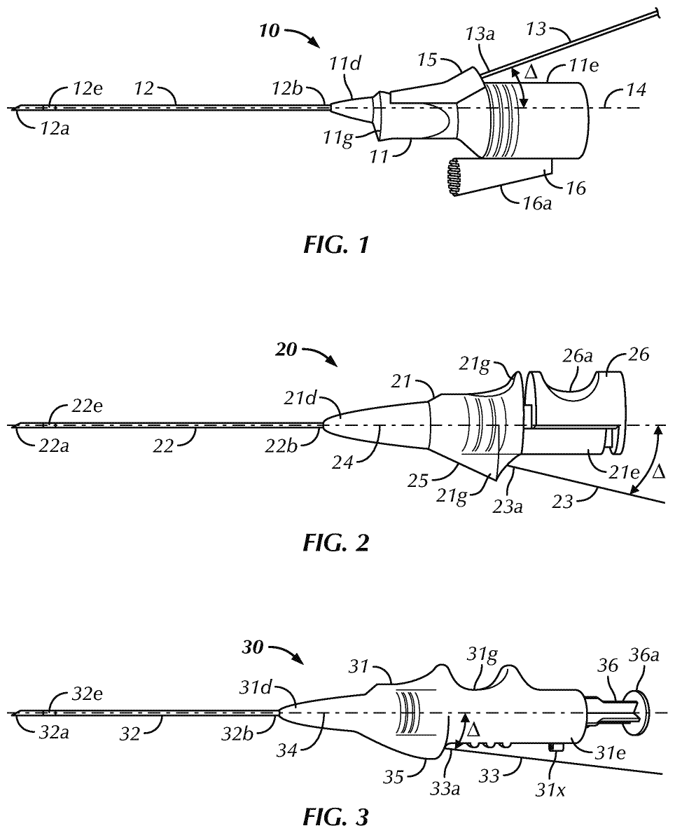

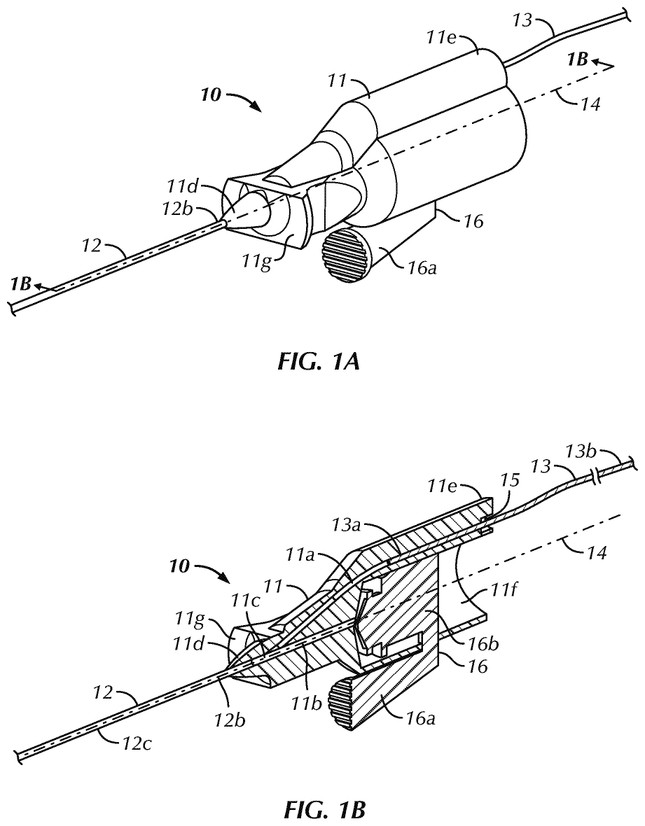

Referring to FIGS. 1-1B, a first preferred embodiment of an access device of the present invention, generally designated 10, includes a hub or body 11, a needle 12 extending from a distal end 11d of the hub 11 and a guidewire 13 movably mounted to the hub 11. The hub 11 includes a guidewire channel 11a and a plunger channel or auxiliary channel 11b that extend into a main channel 11c near the distal end 11d of the hub 11. A proximal end 12b of the needle 12 is attached to the distal end 11d of the hub 11 and is in fluid communication with the main channel 11c. The proximal end 12b of the needle 12 is fixed to the hub 11 in the first preferred embodiment, but is not so limited and may be releasably secured to the hub 11, such as being attached or secured to the hub 11 by a Luer-Lock, threaded connection or other attachment mechanism or method. The first preferred access device 10 also includes a plunger 16 movably mounted in a cylinder 11f that is integrally formed in a proximal end 11e of the hub 11. The plunger 16 has a trigger 16a and a piston 16b that is slidably mounted in the cylinder 11f. The plunger 16 is preferably utilized to draw a vacuum in the main channel 11c for venous access to determine and confirm when a tip 12a of the needle 12 is positioned in a vein or other blood vessel, as is described in greater detail below. The plunger 16 and cylinder 11f may alternatively be utilized to urge medicament or fluid into the patient through the auxiliary channel 11b and the needle 12. The cylinder 11f is preferably in fluid communication with the plunger or auxiliary channel 11b. The guidewire channel 11a preferably extends at an acute angle Δ relative to the longitudinal axis 14.

In the first preferred embodiment, the needle 12, including a needle lumen 12c, the main channel 11c and the plunger channel or auxiliary channel 11b are positioned on and are coaxial with a longitudinal axis 14 of the access device 10. The needle 12, needle lumen 12c, main channel 11c and auxiliary channel 11b are not limited to being positioned on or coaxial with the longitudinal axis 14, but are so configured in the first preferred embodiment. For example, the needle 12, needle lumen 12c and main channel 11c may be positioned on or extend substantially parallel to the longitudinal axis 14 and the auxiliary channel 11b may extend at an angle relative to the longitudinal axis 14. The needle lumen 12c preferably extends between the tip 12a and the proximal end 12b. The needle lumen 12c is in fluid communication with the main channel 11c to facilitate blood flow from the tip 12a into the main channel 11c.

In the first preferred embodiment, a guidewire seal or sealing mechanism 15 is preferably positioned near a proximal end of the guidewire channel 11a near the proximal end 11e of the hub 11 that sealingly engages the guidewire 13 in a mounted configuration. The sealing mechanism 15 may alternatively be positioned proximate a transition between the guidewire channel 11a and the main channel 11c to generally limit or prevent fluid flow into the guidewire channel 11a from the main channel 11c, but also permitting the guidewire 13 to move through the sealing mechanism 15. In the initial configuration, the guidewire 13 may be positioned in the guidewire channel 11a such that a tip of a front end portion 13a of the guidewire 13 is positioned proximally relative to the sealing mechanism 15 and is advanced through the sealing mechanism 15 during use. When the guidewire seal 15 engages the guidewire 13 a seal is preferably created permitting a partial vacuum to be created in the main channel 11c, the guidewire channel 11a and the plunger channel or auxiliary channel 11b, as is described in greater detail below. In addition, the guidewire seal or sealing mechanism 15 generally prevents fluid from flowing past the seal 15 through the guidewire channel 11a, such as preventing blood to flow out of the hub 11 through the guidewire channel 11a during use. The guidewire seal or sealing mechanism 15 is preferably constructed of an elastic material that permits the guidewire 13 to extend therethrough and collapses on itself when the guidewire 13 is removed from the guidewire channel 11a, although the seal 15 is not so limited. The seal 15 may alternatively be constructed to seal around the guidewire 13 when it is inserted into the guidewire channel 11a and generally not seal the guidewire channel 11a when the guidewire 13 is removed from the guidewire channel 11a.

The guidewire 13 includes the front end portion 13a and a rear end portion 13b. The front end portion 13a is preferably pre-loaded into the guidewire channel 11a in an initial configuration (FIGS. 1-1B) such that the guidewire 13 is arranged for quick insertion into the patient when the tip 12a is in the vessel, as is described in greater detail below. The rear end portion 13b may be secured relative to the hub 11 or may extend freely from the hub 11 in the initial configuration.

The first preferred access device 10 can be used to gain access to blood vessels, such as the lumens of arteries and veins. The cannula needle 12 is connected or secured to the hub 11 and the guidewire 13 is pre-loaded onto or into the hub 11, preferably in sterile packaging (not shown) associated with the access device 10. The access device 10 may be supplied to a user as a kit, sterile packaged with the hub 11, needle 12 and guidewire 13 pre-loaded in the hub 11. The hub 11 preferably has features, including the main channel 11c, the guidewire channel 11a and the plunger channel or auxiliary channel 11b, that allow for fluid to pass through the needle 12, into the hub 11 and out of the hub 11. The guidewire 13 is preferably pre-loaded such that it can easily be advanced through the needle 12 and into the lumen of a vessel when properly placed. The access device 10 is preferably designed such that the guidewire 13 is slidable through the hub 11, thereby allowing the guidewire 13 to be left in the vessel while the needle 12 and hub 11 are completely removed from the area adjacent the patient during the procedure by backing the hub 11 and needle 12 proximally away from and off of the guidewire 13.

In the first preferred embodiment, the hub 11 includes the single needle 12 secured or fixed thereto, but is not so limited and multiple needles may be secured to the hub 11 to secure access to the blood vessel by inserting multiple needles into the patient. In addition, the needle 12 may be removably mountable to the hub 11, such as by employing a Luer-Lock connection. The needle 12 is also preferably echogenic or includes an echogenic portion 12e in an area approximately one-half to one centimeter (½-1 cm) from the tip 12a to permit use of the needle 12 with ultrasound for visualization. The echogenic portion 12e may be integrally formed with the needle 12, applied to the needle 12 at the echogenic portion 12e or otherwise positioned at the echogenic portion 12e for visualization purposes. The echogenic portion 12e may alternatively be comprised of a radiopaque marker attached or applied to the needle 12 for visualization using radiant energy techniques and mechanisms, such as x-ray. The echogenic portion 12e may also be positioned at or on the tip 12a. The needle 12 is not limited to including the echogenic portion 12e and may be configured without the echogenic portion 12e, but the echogenic portion 12e is preferred for visualization purposes and may be utilized with any of the preferred access devices 10, 20, 30, 40, 50, 60, 70, 80, 90, 110, 120 described herein. The needle 12 may also include depth markings on an external surface to provide a visual indication of the depth of the tip 12a during insertion into the patient.

The first preferred access device 10 also includes the main channel 11c, the needle lumen 12c and the plunger channel or auxiliary channel 11b positioned generally on or coaxial with the longitudinal axis 14. The main channel 11c and plunger or auxiliary channel 11b are not limited to being positioned on or coaxial with the longitudinal axis 14, as is described in further detail herein.

The guidewire channel 11a of the first preferred embodiment is spaced from and extends into the main channel 11c at an acute angle Δ relative to the longitudinal axis 14, but is not so limited, as is also described in further detail herein. The guidewire channel 11a may be positioned generally along or substantially coaxial with the longitudinal axis 14 with the plunger channel or auxiliary channel 11b spaced from and extending toward the longitudinal axis 14.

The first preferred access device 10 facilitates the access procedure, but within a single, integrated device, which is an improvement over known prior art devices and systems. The hub 11 is designed such that it is integral with or connected to the needle 12, the plunger 16, and the pre-loaded guidewire 13, all with the internal fluidic pathways or channels 11a, 11b, 11c formed in the hub 11. This preferred configuration allows for simultaneous use of the built-in plunger 16 and pre-loaded guidewire 13. The guidewire seal or sealing mechanism 15 facilitates drawing a vacuum in the internal fluidic pathways or channels 11a, 11b, 11c and in the needle 12, preferably at least in the needle lumen 12c, the main channel 11c and the plunger channel 11b, to quickly draw blood from a vessel when penetrated by the tip 12a. The use of the plunger 16 to draw the vacuum is particularly preferred for gaining access to a vein, where blood pressures are typically lower than in an artery and the vacuum urges blood out of the vein and into the access device 10, thereby confirming the tip 12a is positioned in the lumen of the vein.

In operation, the first preferred access device 10 is packaged in a sterile package or kit with the guidewire 12 loaded in the guidewire channel 11a. The access device 10 is removed from the package and the tip 12a is positioned by the physician or medical professional adjacent the patient's skin near an anatomical region where a predetermined vessel should be located for a particular procedure. The tip 12a is inserted into the skin and the physician or medical professional visually inspects the needle 12 and hub 11 waiting for blood to appear in the needle 12 or channels 11a, 11b, 11c as the tip 12a is urged into the patient. In certain procedures, typically arterial access, the pressure in the blood vessel forces blood flow into the needle 12 and channels 11a, 11b, 11c. Alternatively, the physician or medical professional may draw a vacuum in the needle 12 and channels 11a, 11b, 11c once the tip 12a is inserted into the patient's soft tissue to facilitate drawing of blood into the needle once a blood vessel is punctured by the tip 12a and the tip 12a is positioned in the blood vessel. Drawing the vacuum is typically utilized when accessing a vein with generally lower blood pressure than an artery. The guidewire seal 15 facilitates drawing the vacuum by sealing the guidewire channel 11a. The guidewire channel 11a is preferably sealed or partially sealed when the outside surface of the guidewire 13 is engaged by the seal 15 with the guidewire 13 extending through the seal 15. The seal 15 also preferably self-heals or collapses when the guidewire 13 is removed to substantially prevent fluid from flowing out of the guidewire channel 13. Once access is gained to the blood vessel, the needle 12 and hub 11 slide proximally along the longitudinal axis 14 away from the patient, while the guidewire 13 is retained in the patient and, particularly the lumen of the blood vessel. The guidewire 13 is subsequently used to guide instruments or implants into the vessel for further procedures.

Some access procedures are performed without the plunger 16, using only the hub 11, the needle 12 and the guidewire 13, typically for arterial access. The needle 12 is inserted into the vessel and the blood pressure present in the vessel is sufficient to cause the blood to flow out of the needle 12 as an indication of access gained, such as the tip 12a being positioned in the artery. The pre-loaded guidewire 13 is then inserted through the needle 12 and into the vessel, without requiring the physician or medical technician to reach for and insert the guidewire 13 into the guidewire channel 11a and the main channel 11c because of the pre-loading. The guidewire 13 is preloaded in that a front end portion 13a of the guidewire 13 is at least partially positioned in the guidewire channel 11a in an initial configuration (FIGS. 1, 1A and 1B). The guidewire 13 is urged into the appropriate vessel and the needle 12 and hub 11 are then preferably removed over the guidewire 13 away from the patient with the guidewire 13 remaining in the vessel. The guidewire 13 is then utilized to guide additional devices or testing apparatus to the vessel.

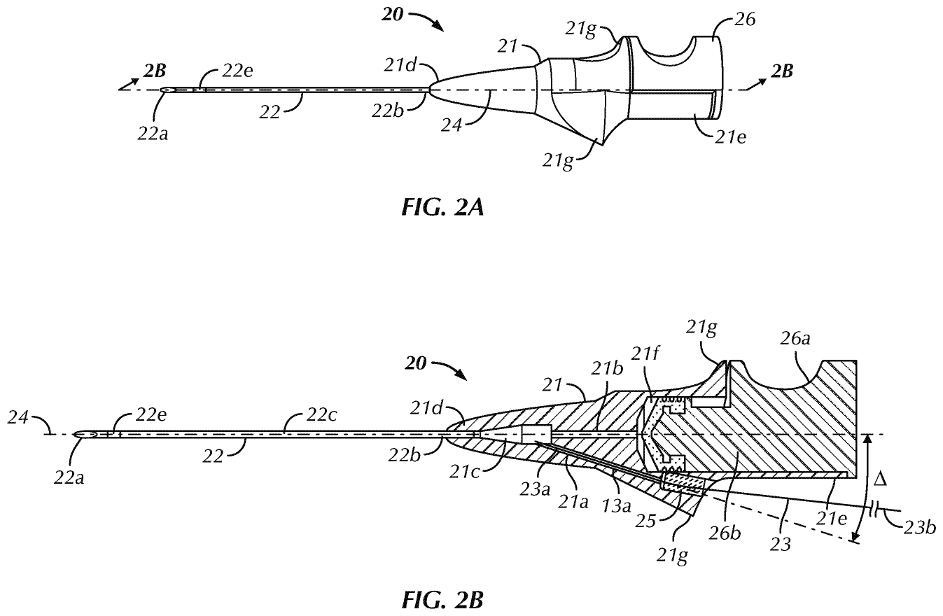

Referring to FIGS. 2-2B, a second preferred access device 20 has a similar construction to the first preferred access device 10 and like reference numbers are utilized to identify like features of the second preferred access device 20 with a number “2” prefix replacing the “1” prefix to distinguish the features of the access device 10 of the first preferred embodiment from the access device 20 of the second preferred embodiment.

The access device 20 of the second preferred embodiment includes a slidable plunger 26 with a finger groove comprising the trigger 26a and the hub 21 includes complementary finger grooves 21g that may be grasped by the user to operate the plunger 26. Similar to the first preferred access device 10, the second preferred access device 20 includes the plunger channel 21b, the main channel 21c, the auxiliary channel 21a and the needle 22 with the needle lumen 22c. The plunger channel 21b, main channel 21c and the needle lumen 22c are preferably positioned coaxial with the longitudinal axis 24 with the guidewire or auxiliary channel 21a extending at an acute angle Δ toward the longitudinal axis 24 into the main channel 21c. The hub 21 includes the distal end 21d, the proximal end 21e, the cylinder 21f and the finger grooves 21g. The needle has the tip 22a, the proximal end 22b, the needle lumen 22c and the echogenic portion 22e proximate the tip 22a. The guidewire 23 includes the front end portion 23a that is preferably pre-loaded into the guidewire channel 21a in the initial configuration (FIG. 2B). The second preferred access device 20 operates substantially the same the first preferred access device 10 to gain access to the bodily vessel, position the guidewire 23 in the vessel and guide subsequent instruments to the vessel along the appropriately positioned guidewire 23.

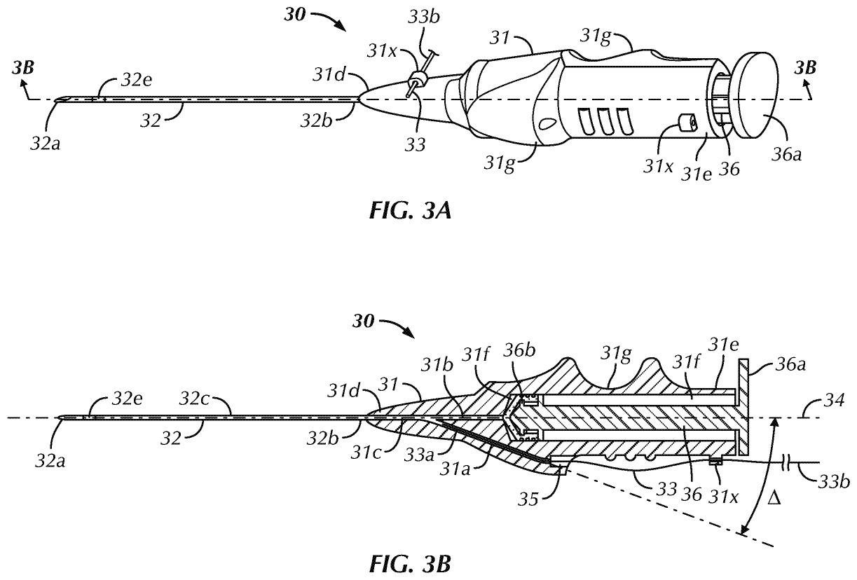

Referring to FIGS. 3-3B, a third preferred access device 30 has a similar construction to the first preferred access device 10 and like reference numbers are utilized to identify like features of the third preferred access device 30 with a number “3” prefix replacing the “1” prefix to distinguish the features of the access device 10 of the first preferred embodiment from the access device 30 of the third preferred embodiment.

The access device 30 of the third preferred embodiment includes a generally conventional plunger 36 with a trigger 36a at a distal end. The physician or medical professional is able to grasp the hub 31 at the finger grooves 31g to insert the needle 32 into the patient and manipulate the plunger 36 to draw a vacuum, as was described above. The generally conventional plunger 36 provides familiarity of use for the physician or medical professional when compared to a conventional syringe. The hub 31 includes the guidewire channel 31a, the plunger or auxiliary channel 31b, the main channel 31c, the distal end 31d, the proximal end 31e and the cylinder 31f. The needle 32 includes the tip 32a, the proximal end 32b, the needle lumen 32c and the echogenic portion 32e. The needle lumen 32c, the main channel 31c and the plunger channel 31b are positioned on and coaxially with the longitudinal axis 34. The hub 31 may also include guidewire clips 31x that are configured to guide or secure the guidewire 33 relative to the hub 31. The front end portion 33a of the guidewire 33 is preferably pre-loaded into the guidewire channel 31a in the initial configuration when the tip 32a is inserted into the patient for relatively quick extension into the vessel. The guidewire clips 31x may guide the guidewire 33 into the guidewire channel 31a or may secure the guidewire 33 relative to the hub 31, such as securing the rear end portion 33b relative to the hub 31 to reduce interference or uncontrolled movement of the rear end portion 33b relative to the hub 31 during the access procedures. The plunger 36 may be urged rearwardly when inserting the tip 32a to draw a vacuum in the main channel 31c, the needle lumen 32c, the cylinder 31f and the plunger channel 31b to draw blood from the vessel when the tip 32a is positioned in the vessel, particularly when attempting to gain access to a vein.

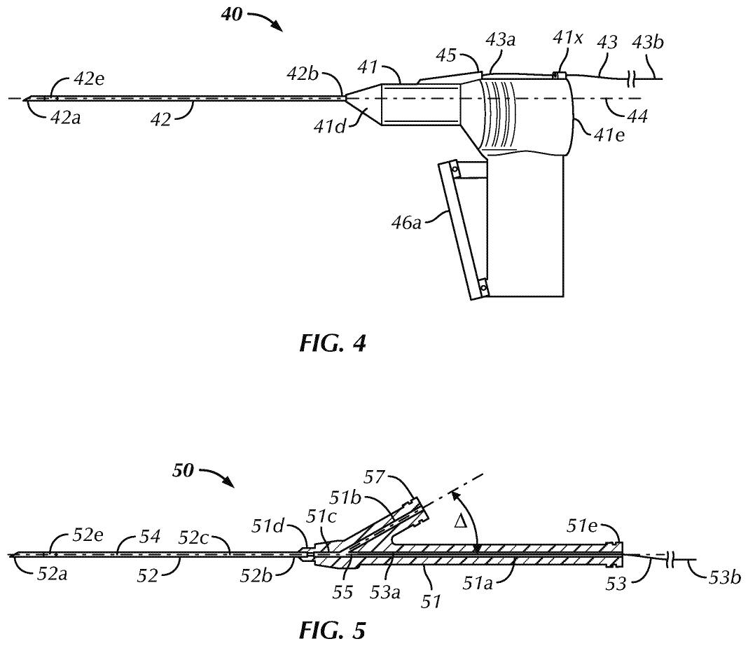

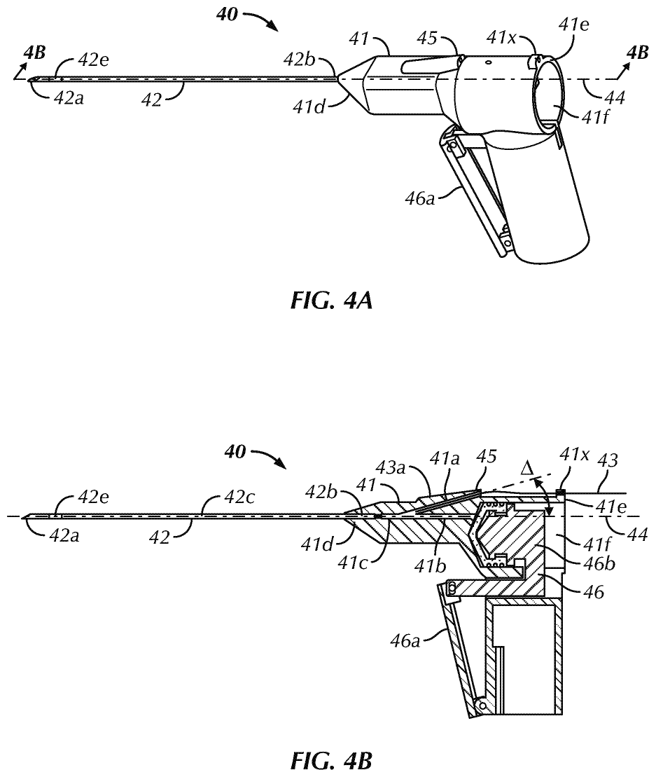

Referring to FIGS. 4-4B, a fourth preferred access device 40 has a similar construction to the first preferred access device 10 and like reference numbers are utilized to identify like features of the fourth preferred access device 40 with a number “4” prefix replacing the “1” prefix to distinguish the features of the access device 10 of the first preferred embodiment from the access device 40 of the fourth preferred embodiment. The hub 41 of the fourth preferred embodiment includes the guidewire channel 41a, the plunger channel 41b, the main channel 41c, the distal end 41d, the proximal end 41e and the cylinder 41f. The needle 42 includes the tip 42a, the proximal end 42b, the needle lumen 42c and the echogenic portion 42e for visualization. The needle lumen 42c, the main channel 41c and the plunger channel 41b are positioned on and are coaxial with the longitudinal axis 44, but are not so limited. The auxiliary channel 41a extends from the longitudinal axis 44 at the acute angle Δ. The fourth preferred access device 40 also includes the guidewire clip 41x that guides the guidewire 43 into the guidewire channel 41a proximate the outer surface of the hub 41. The guidewire seal 45 is positioned near a proximal end of the guidewire channel 41a to limit or prevent flow of fluid through the guidewire channel 41a and to promote the vacuum or partial vacuum created by the plunger 46, as is described below.

In the fourth preferred embodiment, the access device 40 includes a hinged trigger 46a mounted to a lower barrel portion of the hub 41. The hinged trigger 46a is also secured to the slidable piston 46b such that urging the trigger 46a toward the lower barrel of the hub 41 causes the piston 46b to slide away from the main channel 41c in the cylinder 41f. When the tip 42a is positioned in the patient's soft tissue, actuating the trigger 46a draws at least a partial vacuum in the main channel 41c, the plunger channel or auxiliary channel 41b, the cylinder 41f and the guidewire channel 41a to draw blood into the needle 42 when the tip 42a is positioned in the blood vessel, preferably a vein. The access device 40 otherwise is configured and operates similarly to the preferred access devices 10, 20, 30 of the first, second and third preferred embodiment, as would be apparent to one having ordinary skill in the art based on a review of the present disclosure.

Referring to FIGS. 1-4B, the first, second, third and fourth preferred access devices 10, 20, 30, 40 facilitate generation of a vacuum through the needle 12, 22, 32, 42, typically for drawing blood flow during venous access. The first through fourth preferred access devices 10, 20, 30, 40 also include the pre-loaded and sealed guidewires 13, 23, 33, 43, in a single device or ready to use kit for conducting the access procedures described herein. The first, second, third and fourth preferred access devices 10, 20, 30, 40 provide an advantage in that the devices 10, 20, 30, 40 do not require the user to acquire separate components during the time-critical access procedure. The first, second, third and fourth preferred access devices 10, 20, 30, 40 also simplify the procedure by providing the pre-loaded guidewire 13, 23, 33, 43 that can be easily advanced into the main channel 11c, 21c, 31c, 41c and through the needle lumen 12c, 22c, 32c, 42c out of the tip 12a, 22a, 32a, 42a, without excessive handling of a needle 12, 22, 32, 42 that is delicately and precisely placed in the blood vessel. The first, second, third and fourth preferred access devices 10, 20, 30, 40 are also specifically adapted for access in a vein, which typically has lower blood pressures than an artery, but is not so limited and may also be utilized for access procedures targeting arteries.

The plungers 16, 26, 36, 46 and mechanisms for drawing a vacuum in the needle 12, 22, 32, 42 have all been presented as a mechanical structures and mechanisms in the first, second, third and fourth preferred access devices 10, 20, 30, 40. These vacuum drawing devices or systems could alternatively be constructed from a collapsible bulb (not shown) (akin to a pipet), a vacuum chamber that is advanced and pierced during use to generate and draw a vacuum through the needle 12, 22, 32, 42, as well as alternative orientations and configurations of the plungers 16, 26, 26, 46 represented in the above-described first, second, third and fourth preferred access devices 10, 20, 30, 40.

Referring to FIG. 5, a fifth preferred access device 50 has a similar construction to the first, second, third and fourth preferred access devices 10, 20, 30, 40 and like reference numbers are utilized to identify like features of the fifth preferred access device 50 with a number “5” prefix replacing the “1,” “2,” “3” and “4” prefixes to distinguish the features of the access devices 10, 20, 30, 40 of the first, second, third and fourth preferred embodiments from the access device 50 of the fifth preferred embodiment.

The access device 50 of the fifth preferred embodiment does not include an integral plunger, but has a flash port 57 integrally formed with the hub 41 at the distal end of the auxiliary channel 51b that extends at the acute angle Δ away from the longitudinal axis 54. The auxiliary channel 51b preferably extends at the acute angle Δ between the main channel 51c and the flash port 57. The flash port 57 is preferably utilized in arterial access procedures and permits flow of blood out of the flash port 57 when the tip 52a is positioned in the artery. When the tip 52a is positioned in the lumen of the artery, the blood typically flows through the needle lumen 52c, into the main channel 51c, through the auxiliary channel 51b and out of the flash port 57 to provide a visual indication that the tip 52a is in the lumen. The blood may also enter the guidewire channel 51a, but is generally blocked from flowing through the guidewire channel 51a by the pre-loaded guidewire 53 and the guidewire seal 55 between the main channel 51c and the guidewire channel 51a. The flash port 57 also preferably includes a Luer-Lok connector for selective engagement of a conventional syringe to the flash port 57 such that a vacuum may be drawn within the hub 51, as is described above. The Luer-Lok connector included on the flash port 57 can also be connected to a pressure-monitoring device as described in greater detail below. The hub 51 also includes the main channel 51c, the guidewire channel 51a and the auxiliary channel 51b that extends from the main channel 51c to the flash port 57. The main channel 51c, the needle lumen 52c and the guidewire channel 51a are preferably positioned on and are coaxial with the longitudinal axis 54, but are not so limited and may be otherwise arranged such that the guidewire 54 may be fed into the needle 52 and the blood vessel lumen in operation.

The guidewire 53 is preferably pre-loaded into the hub 51 such that the physician or medical professional is not required to reach for and feed the guidewire 53 into the hub 51, as was described above in the prior art systems. In the fifth preferred embodiment, the guidewire 53 is pre-loaded with the front end portion 53a substantially on the longitudinal axis 54 such that the guidewire 53 is slidable through the hub 51 substantially along the longitudinal axis 54 through the seal 53a, the main channel 51c and the needle lumen 52c. Such alignment of the guidewire 53 along the longitudinal axis 54 reduces the bending of the guidewire 53 as it is fed into and through the hub 51, particularly when compared to the path of the guidewires 13, 23, 33, 43 of the first, second, third and fourth preferred embodiments through the hubs 11, 21, 31, 41. The pre-loaded guidewire 53 preferably extends out of the proximal end 51e of the hub 51 such that the medical professional is able to urge the guidewire 53 may be pushed along the longitudinal axis 54 through the seal 55 and into the vessel. The needle 52 preferably includes the echogenic portion 52e for visualization during insertion.

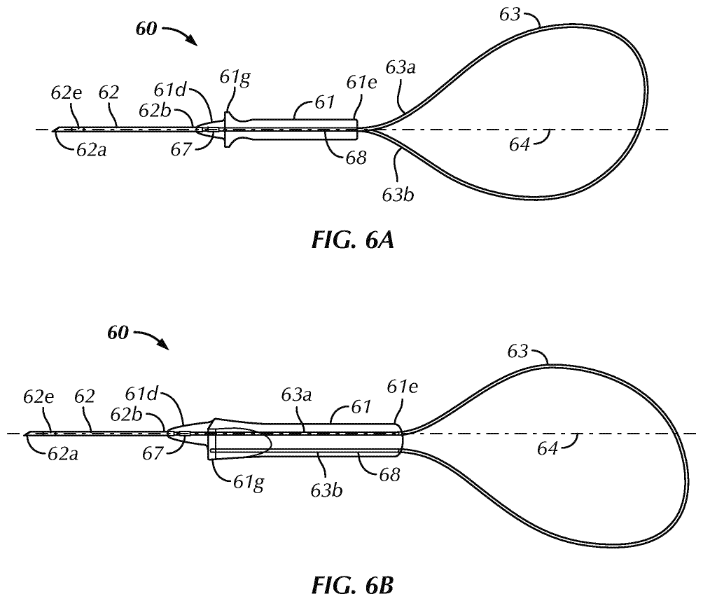

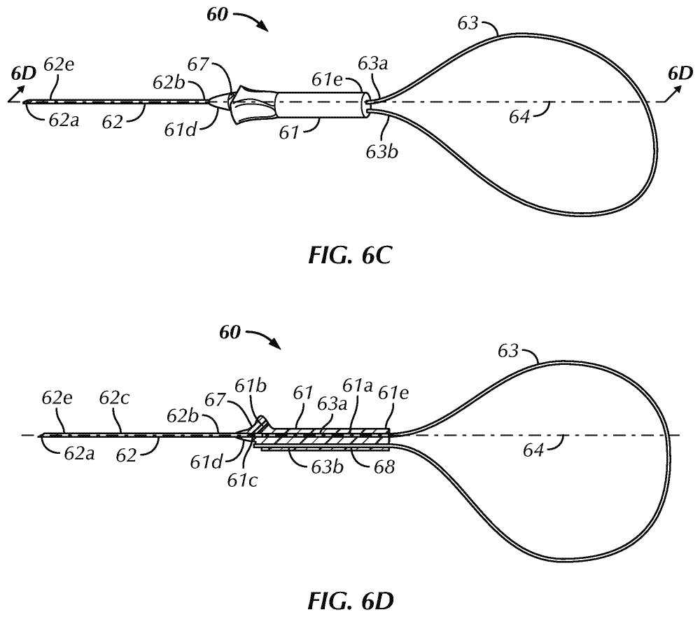

Referring to FIGS. 6A-6D, a sixth preferred access device 60 has a similar construction to the fifth preferred access device 50 and like reference numbers are utilized to identify like features of the sixth preferred access device 60 with a number “6” prefix replacing the “5” prefix to distinguish the features of the access device 50 of the fifth preferred embodiment from the access device 60 of the sixth preferred embodiment.

The access device 60 of the sixth preferred embodiment includes a flash port 67 near the distal end 61d of the hub 61 and finger grooves 61g. The preferred flash port 67 does not include the Luer-Lok of the fifth preferred embodiment, but is not so limited and may be adapted to include the Luer-Lok for attachment of a syringe for drawing a vacuum. The hub 61 preferably includes the auxiliary channel 61b that extends from the main channel 61c to the flash port 67 and the guidewire channel 61a that extends from the main channel 61c out of the proximal end 61d of the hub 61 to accommodate the guidewire 63. The hub 61 of the sixth preferred embodiment also includes a guidewire holder 68 that opens at the proximal end 61d and has a closed or open end near the finger grooves 61g near the distal end 61d of the hub 61. The guidewire holder 68 secures a rear end portion 63b of the guidewire 63 in the hub 61 when the front end portion 63a is secured in the guidewire channel 61a. Securing the front and rear end portions 63a, 63b of the guidewire 63 to the hub 61 facilitates the pre-loading of the guidewire 63 and the flash port 67 allows for the blood flow to clearly present to the user to align the guidewire 63 when the tip 62a is in the blood vessel for advancement of the pre-loaded guidewire 63. The guidewire holder 68 preferably extends substantially parallel to the longitudinal axis 64 from the distal end 61d to the proximal end 61e of the hub 61. The needle lumen 62c, the main channel 61c and the auxiliary channel 61a are preferably positioned on and are coaxial with the longitudinal axis 64. The guidewire holder 68 may be utilized with any of the preferred hubs 11, 21, 31, 41, 51, 61, 71, 81, 91, 111 described herein to secure the rear end portion 63b of the guidewire 63 in the initial configuration.

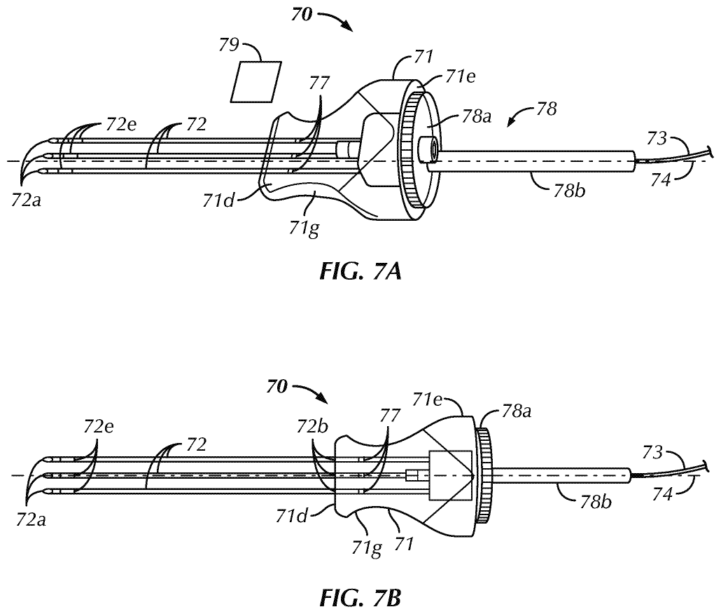

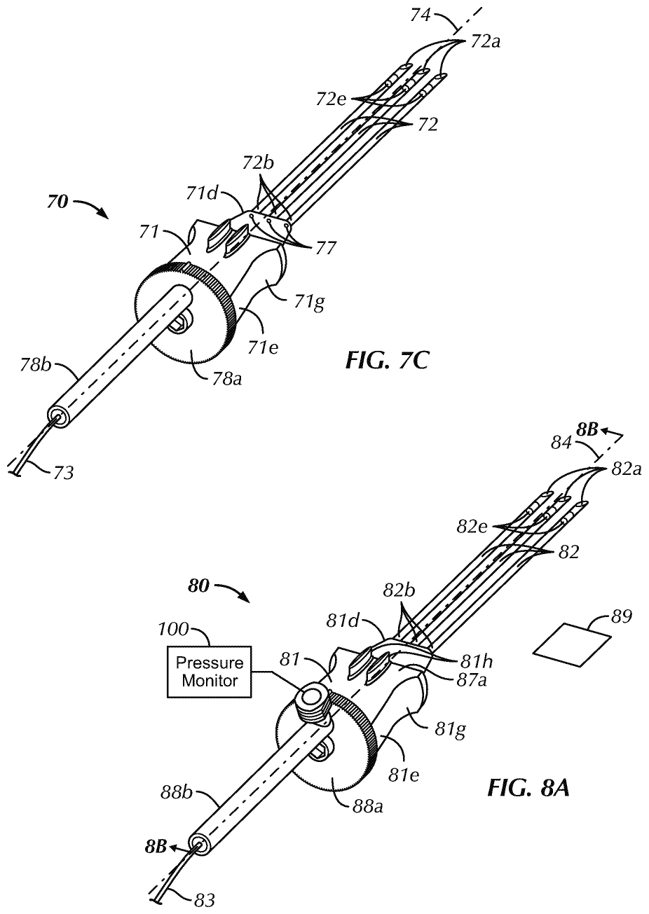

Referring to FIGS. 7A-7C, a seventh preferred access device 70 has a similar construction to the sixth preferred access device 60 and like reference numbers are utilized to identify like features of the seventh preferred access device 70 with a number “7” prefix replacing the “6” prefix to distinguish the features of the access device 60 of the sixth preferred embodiment from the access device 70 of the seventh preferred embodiment.

The access device 70 of the seventh preferred embodiment includes three (3) needles 72 and associated tips 72a for insertion into the patient to access a blood vessel. The three (3) needles 72 preferably define a needle plane 79 upon which each of the needles 72 and the associated tips 72a are positioned. The needles 72 are also each, preferably positioned parallel to or coaxial with the longitudinal axis 74. The seventh preferred access device 70 operates similarly to the device of US Patent Application Publication No. 2014/0249504 directed to a “Vascular Access System and Methods of Use,” the contents of which are incorporated herein by reference in their entirety, without use of the described shuttle. Each of the three (3) needles 72 is associated with a flash port 77 that provides a visual indication to the user when one of the tips 72a has punctured and is positioned within the lumen of the target blood vessel, typically an artery when utilized with the seventh preferred embodiment. Each of the flash ports 77 is preferably associated with an auxiliary port (not shown), each of which is also associated with a main channel (not shown) that is in fluid communication with the individual needles 72, respectively. The hub 71 also includes three guidewire channels (now shown) associated with each of the main channels that provide a pathway for insertion of the guidewire 73 into the main channels. The hub 71 has the proximal end 71e and the distal end 71d. The proximal ends 72b of the needles 72 are connected to the distal end 71d of the hub 71. Each of the needles 72 also preferably includes the echogenic portion 72e for visualization during use.

The seventh preferred access device 70 also includes a selector dial 78a and a guidewire tube 78b that comprise the guidewire holder 78. The selector dial 78a and guidewire tube 78b are pivotably mounted to the proximal end 71e of the hub 71 and secure or pre-load the guidewire 73 to the hub 71. The guidewire 73 is pre-loaded with its proximal end in the guidewire tube 78b. Once the needles 72 are inserted into the patient and one of the needles 72 visually indicates blood flow out of the flash port 77, the user is able to pivot the selector dial 78a such that the guidewire tube 78b and the guidewire 73 are aligned with the needle 72 that shows the flash. The user is then able to slide the guidewire 73 into the associated main channel, into the needle 72 and into the vessel. The access device 70 may then slide distally away from the patient, such that the guidewire 73 remains in the blood vessel. A device or instrument may then slide over the guidewire 73 into the vessel for subsequent treatment of the patient. The seventh preferred access device 70 provides advantages of multiple needles 72 to increase the likelihood that access is gained to the vessel in a single operation with the pre-loaded guidewire 73 that that is quickly aligned with the appropriate needle 72.

The access devices 50, 60, 70 of the fifth, sixth and seventh preferred embodiments provide the advantage of a pre-loaded guidewire in combination with the flash port 57, 67, 77 that is designed to clearly, quickly, and consistently present the flash of blood to the user. The flash ports 57, 67, 77 provide small fluidic pathways that allow the flow of blood to immediately exit the hub 51, 61, 71 via a relatively high velocity fluid whose trajectory can be clearly and easily observed by the user when the tips 52a, 62a, 72a are positioned in the vessel. This provides the user with an almost instantaneous indication that the needle 52, 62, 72 is correctly placed into the lumen of the vessel, thereby preventing unwanted over-travel or loss of access due to excessive handling.

The seventh preferred access device 70 includes the multiple needles 72 to increase the likelihood of gaining access on the first try or procedure when targeting the vessel. The seventh preferred access device 70 also includes the pre-loaded guidewire 73 and the guidewire holder 78 with the selector dial 78a and the guidewire tube 78b that can rotate/translate relative to the needle 72 of choice. The seventh preferred access device 70 is not limited to inclusion of the guidewire holder 78 with the selector dial 78a comprising the guidewire holder 78, which may alternatively be comprised of a selector that translationally shifts the distal end of the guidewire 73 into alignment with the appropriate needle 72 or nearly any mechanism that is able to secure the guidewire 73 for pre-loading and align the guidewire 73 with the appropriate needle 72 once one of the tips 72a is positioned in the vessel. The guidewire holder 78 may also be comprised of a mechanism that holds individual guidewires 73 for each of the multiple needles 72 incorporated into or associated with the hub 71. The guidewire holder 78 of the seventh preferred embodiment could utilize the rotational mechanism, including the selector dial 78a and the guidewire tube 78b described above, but may also be comprised of translational mechanisms or contain multiple guidewires 73, rather than a mechanism that moves the wire to the desired needle 72.

The flash ports 57, 67, 77 of the fifth, sixth and seventh preferred embodiments could be configured in any number of ways and utilize different geometries to alter how the blood flash is presented to the user. The flash ports 57, 67, 77 could also contain features that allow the connection of a syringe or other similar devices for drawing a vacuum, such as the Luer-Lock of the flash port 57 of the fifth preferred embodiment.

Referring to FIGS. 8A and 8B, an eighth preferred access device 80 has a similar construction to the seventh preferred access device 70 and like reference numbers are utilized to identify like features of the eighth preferred access device 80 with a number “8” prefix replacing the “7” prefix to distinguish the features of the seventh preferred access device 70 from the access device 80 of the eighth preferred embodiment.

The eighth preferred access device 80 is similar to the seventh preferred access device 70, but is configured to utilize a pressure monitor 100 (either external or built-in) to monitor pressure in the fluid pathway, including in the auxiliary channel 81b, the main channel 81c and within the needle lumen 82c of the needle 82. The preferred pressure monitor 100 visually indicates to the user when the tip 82a of the needle 82 is correctly position within the lumen of the vessel.

The pressure monitor 100 preferably detects and visually indicates pressure in the channels 81a, 81b, 81c and the needle lumen 82c of the needle 82 due to the blood pressure present in these areas after the tip 82a is positioned in the vessel. The pressure monitor 100 may visually indicate the pressure through mechanical or electronic means, such as by a mechanical gauge that provides an indication at a predetermined pressure, a go or no-go mechanical indicator or a video or display screen that visually indicates a pressure level. The pressure monitor 100 could represent pressure via a direct display showing pressure of either the blood itself or through the motion of a gauge (e.g., dial, pin, fluid column, etc.). The pressure monitor 100 may include an electronic pressure transducer that indicates the presence of pressure through an electronic display (e.g., lights, digital display, audio, etc.). The pressure monitor 100 may be configured and adapted for use with any of the preferred hubs 11, 21, 31, 41, 51, 61, 71, 81, 91, 111 described herein for monitoring, detecting and measuring pressure, preferably in the main channel 11c, 21c, 31c, 41c, 51c, 61c, 71c, 81c, 91c, 111c, the needle lumen 12c, 22c, 32c, 42c, 52c, 62c, 72c, 82c, 92c, 111c and the auxiliary channel 11b, 21b, 31b, 41b, 51b, 61b, 71b, 81b, 91b, 111b.

The eighth preferred access device 80 includes a first series of three (3) flash ports 87a positioned on a top surface of the hub 81 between and laterally spaced from dividers 81h that extend from the tip surface of the hub 81. The dividers 81h provide a block to blood that flows out of each of the individual first series of flash ports 87a so that a user is able to detect which of the first series of flash ports 87a blood is flowing from when one of the tips 82a is positioned in the lumen of the vessel. The dividers 87h generally prevent lateral flow or splashing of the blood once it flows out of the individual one of the first series of flash ports 87a to limit confusion of the user as to which of the first series of flash ports 87a the blood is flowing. In the eighth preferred embodiment, the selector dial 88a also includes a second flash port 87b with a connector, such as a Luer-Lok connector, for selective mounting of the pressure monitor 100. The pressure monitor 100 may be mounted to the second flash port 87b to detect pressure in the auxiliary channel 81b when the guidewire tube 88b is aligned with the needle 82 that is positioned within the lumen of the vessel. The pressure monitor 100 may alternatively be mounted to the first series of flash ports 87a or separate pressure monitors (not shown) may be mounted to each one of the first series of flash ports 87a to detect pressure.

The eighth preferred access device 80 includes the needles 82 with tips 82a, proximal ends 82b and needle lumens 82c. The eighth preferred hub 81 includes the guidewire channel 81a, the auxiliary channel 81b, the main channel 81c, the distal end 81d and the proximal end 81e. Each of the needles 82 preferably include the echogenic portions 82e proximate the tips 82a for visualization.

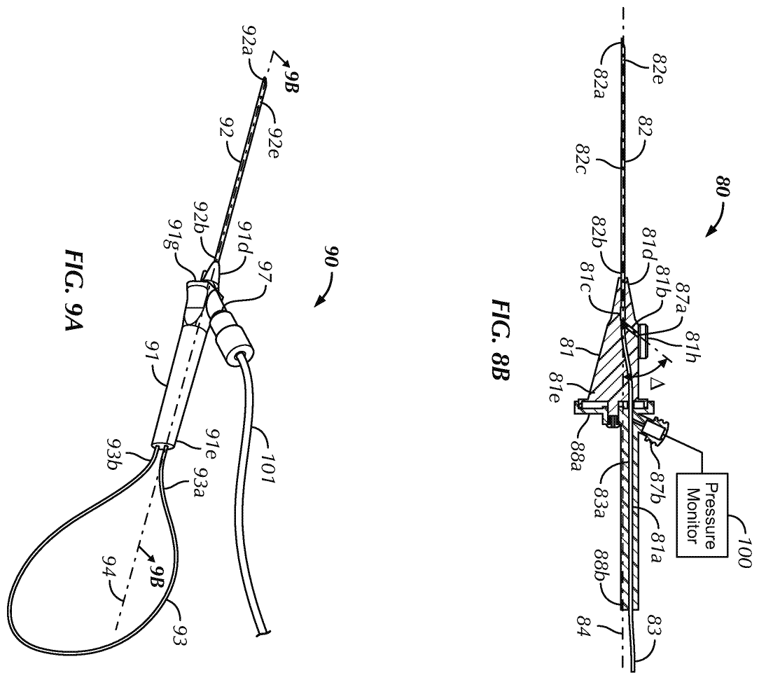

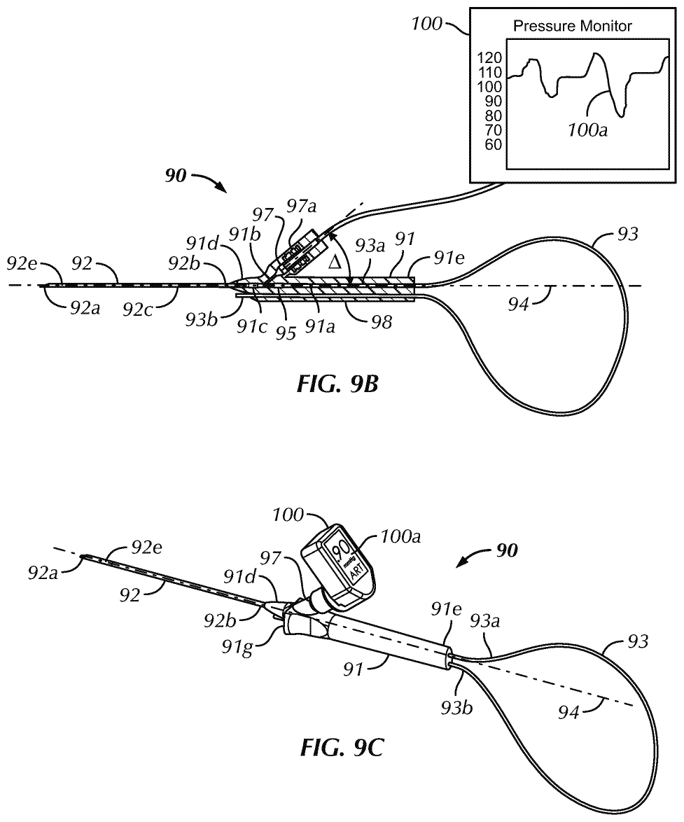

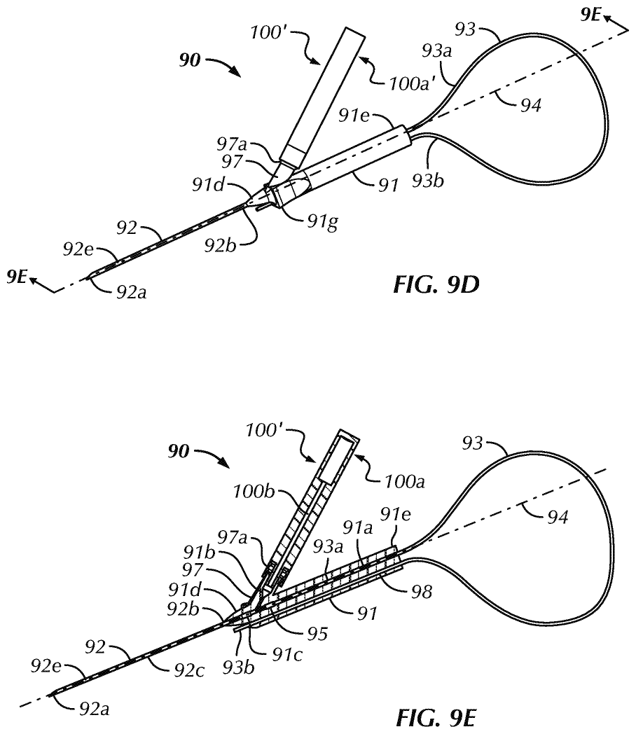

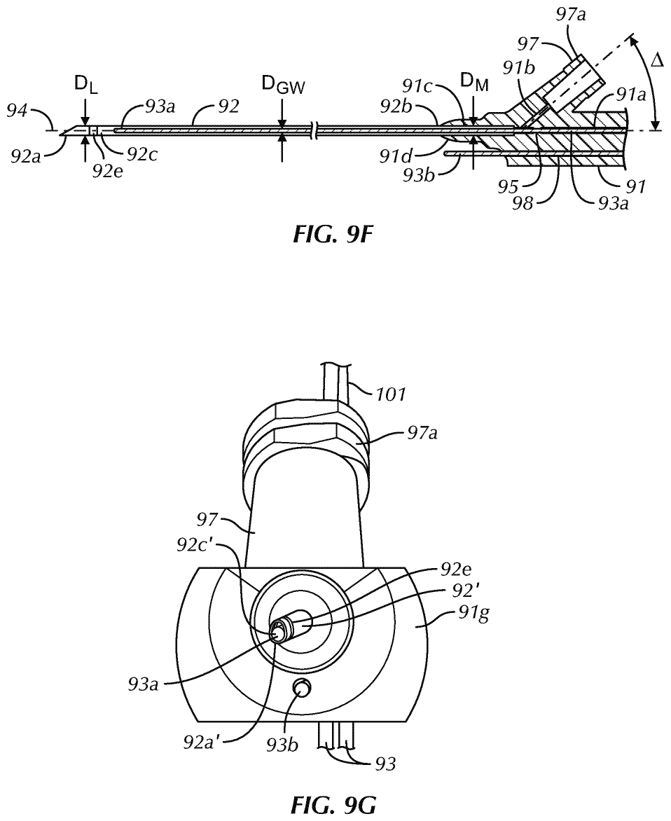

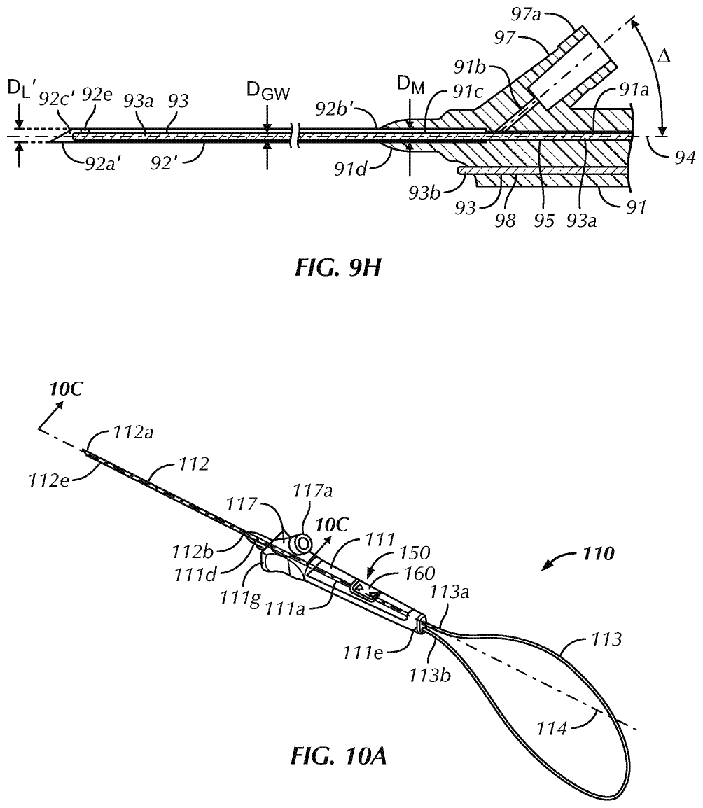

Referring to FIGS. 9A-9C, an ninth preferred access device 90 has a similar construction to the sixth preferred access device 60 and like reference numbers are utilized to identify like features of the ninth preferred access device 90 with a number “9” prefix replacing the “6” prefix to distinguish the features of the sixth preferred access device 60 from the access device 90 of the ninth preferred embodiment.

Referring to FIGS. 9A-9C, the ninth preferred access device 90 is similar to the access device 60 of the sixth preferred embodiment, but includes the flash port 97 with an attachment 97a, such as the a Luer-Lok style connector, a bayonet-style connector, mechanical threads or other attachment mechanism, to engage or releasably mount the pressure monitor 100 to the hub 91. The pressure monitor 100 is utilized to monitor pressure in the auxiliary channel 91b, the main channel 91c and in the needle 92 to detect when the tip 92a is positioned within the vessel. The pressure monitor 100 may be connected directly to the attachment 97a or may be connected to an extension tube 101 that spaces a display or main unit 100a of the pressure monitor 100 from the hub 91.

The ninth preferred access device 90 includes the hub 91, the needle 92 and the guidewire 93. The hub 91 includes the guidewire channel 91a, the auxiliary channel 91b, the main channel 91c, the distal end 91d, the proximal end 91e and the finger grooves 91g that may be grasped by the user during the procedure to control insertion and removal of the hub 91 in operation. The needle 92 includes the tip 92a, the proximal end 92b, the needle lumen 92c and the echogenic portion 92e proximate the tip 92a for visualization. The guidewire 93 includes the front end portion 93a that is pre-loaded into the guidewire channel 91a in the initial configuration and the rear end portion 93b that is pre-loaded into a guidewire holder 98 in the initial configuration. The needle lumen 92c, the main channel 91c and the guidewire channel 91a are preferably positioned on and coaxially with the longitudinal axis 94 or substantially parallel relative to the longitudinal axis 94 in the initial configuration. The auxiliary channel 91b preferably extends at the acute angle Δ relative to the longitudinal axis 94 between the main channel 91c and the flash port 97. The guidewire holder 98 preferably extends parallel to the longitudinal axis 94 and is open at both proximal and distal ends, but is not so limited and may be open at the distal end and closed at the proximal end, as long as the guidewire 93 may be positioned and held in the guidewire holder 98 in the initial configuration. The guidewire channel 91a is not limited to being oriented coaxial or parallel to the longitudinal axis 94 and the auxiliary channel 91b is not limited to extending at the acute angle Δ relative to the longitudinal axis 94. The guidewire channel 91a may extend away from the longitudinal axis 94 at the acute angle Δ, the auxiliary channel 91b may be coaxial or extend parallel to the longitudinal axis 94, both the guidewire channel 91a and the auxiliary channel 91b may extend at angles away from the longitudinal axis 94, both the guidewire channel 91a and the auxiliary channel 91b may extend substantially parallel to the longitudinal axis 94 or the auxiliary and guidewire channels 91b, 91a may be otherwise oriented in the hub 91 so that they are able to perform the preferred functions of the access device 90, as is described herein. The guidewire holder 98 secures the rear end portion 93b of the guidewire 93 during the procedure to limit movement of the rear end portion 93b and prevent flopping of the rear end portion 93b during the procedure. The rear end portion 93b is not limited to being positioned in the guidewire holder 98 in the initial configuration and may extend freely from the hub 91 without being secured to the hub 91.

In both the eighth and ninth preferred embodiments of the access device 80, 90, the display of pressure on the pressure monitor 100 could have a secondary feature that allows the user to directly distinguish between arterial and venous access, as this is typically not a trivial assessment for the user. Accordingly, the pressure monitor 100 may include an indication or indicator on its display 100a that provides a designation to the user regarding whether the pressure indicates access to an arterial lumen or a venous lumen. The indication may be provided based on a relatively consistent pressure being detected by the pressure monitor 100, which typically indicates venous access, or a pulsing pressure or a pulsatile flow being detected by the pressure monitor 100, which typically indicates arterial access.

Referring to FIGS. 9D and 9E, the ninth preferred access device 90 may also include an alternative pressure monitor 100′ mounted to the attachment 97a of the flash port 97. The alternative pressure monitor 100′ has a similar function to the above-described pressure monitors 100 of providing an indication to the user of access to an artery or vein of the patient. The alternative pressure monitor 100′ preferably provides the pressure indication or indication of whether the tip 92a is positioned within an artery or vein passively or without power from an external source. The alternative pressure monitor 100′ or Arterial vs Vein Indication Device (“AVID”) is preferably comprised of a closed tube with a luer-lock connection 100b at its distal end that releasably engages the attachment 97a of the flash port 97. The alternative pressure monitor 100′ is not limited to being releasably attached by the Luer-Lock and may be connected using nearly any releaseable connection that connects the alternative pressure monitor 100′ to the access device 90 to perform the preferred function, as is described herein. The AVID or alternative pressure monitor 100′ is releasably attached to the flash port 97 and when the tip 92a of the needle 92 enters the patient's vessel, preferably an artery or vein, blood flow due to pressure differential flows into the needle 92, the main channel 91c, the auxiliary channel 91b and ultimately into a monitor channel 100b of the AVID device 100′. Due to the trapped air in the monitor channel 100b of the AVID device 100′, when arterial access is gained, the pressure of the blood flow will travel farther or higher into the monitor channel 100b than when venous access is gained and the arterial blood flow will typically indicate pulsatile flow when a top of the flow sequentially moves up and down in the monitor channel 100b. The AVID device 100′ is preferably constructed of a transparent or semi-transparent material such that the user is able to visually detect the blood flow within the monitor channel 100b during use, including the level that the blood flow reaches in the monitor channel 100b and changes in pressure of the blood flow, by observing movement of the top of the blood flow within the monitor channel 100b. The blood flow within the monitor channel 100b generally moves higher in the monitor channel 100b when an artery is access, because the arterial pressure is typically greater, when compared to venous pressure, and the arterial pressure will have greater capacity to compress the trapped air in the monitor channel 100b. Additionally, the pulsatile nature of arterial flow will be visually presented in the AVID device 100′ by a distinct level change or pulsing of the blood in the monitor channel 100b as a result of the relatively small flow diameter within the monitor channel 100b. Accordingly, the body of the AVID device 100′ with its transparent or semi-transparent material acts substantially as a display 100a′ for the user to visually determine whether the tip 92a is within an artery or a vein.

Referring to FIG. 9F, the ninth preferred access device 90 may also be configured such that the guidewire 93 has a smaller outer diameter D_{GW} than the inside diameter D_{M} of the main channel 91c and an inside diameter D_{L} of the needle lumen 92c of the needle 92. The guidewire 93 may be sized and configured to have the smaller outside diameter D_{GW} such that the guidewire 93 may be loaded into the lumen 92c, through the main channel 91c and the guidewire channel 91a in the initial configuration. The larger inside diameter D_{M} of the main channel and inside diameter D_{L} of the needle lumen 92c accommodate the smaller diameter D_{GW} of the guidewire 93. Blood from the pierced artery or vein is able to flow past the guidewire 93 that is at least partially loaded into the lumen 92c and into the auxiliary channel 91b for pressure detection by the pressure monitor 100 or the alternative pressure monitor 100′ or for flash out of the distal end of the auxiliary channel 91b. The blood is able to flow past the guidewire 93 in the needle lumen 92c and the main channel 91c because of the smaller outer diameter D_{GW} of the guidewire 93 compared to the inner diameters D_{M}, D_{L} of the needle lumen 92c and the main channel 91c permits blood flow between the outer surface of the guidewire 93 and the inner surfaces of the lumen 92c and the main channel 91c. The sealing mechanism 95 generally prevents blood flow through and out of the end of the guidewire channel 91a. The sealing mechanism 95 is also preferably self-healing such that the sealing mechanism 95 continues to generally prevent fluid flow therethrough after being pierced by the guidewire 93.

Advancing the guidewire 93 into the lumen 92c of the needle 92 in the initial configuration limits the amount of movement required to insert the guidewire 93 into the vessel during use, specifically, after the tip 92a is positioned in the vessel. In addition, sizing the guidewire 93 such that blood may continue to flow to the pressure monitor 100, the alternative pressure monitor 100′ our out of a flash port after the guidewire 93 is inserted into the vessel provides assurance to the surgeon or medical technician that the tip 92a of the needle 92 has not inadvertently moved out of the vessel during insertion of the guidewire 93. Movement of the guidewire 93 through the needle lumen 92c while the tip 92a is in the vessel can cause the tip 92a to move within the patient and potentially move out of the vessel. Sizing the guidewire 93 to have the smaller outer diameter D_{GW} than the inner diameter D_{L} of the needle lumen 92c and the inner diameter D_{M} of the main channel 91c provides assurance to the surgeon or medical technician that the guidewire 93 is placed in the vessel after insertion of the guidewire 93 to its final location in the patient, because pressure in the vessel can be continuously monitored by the pressure monitor 100 or the alternative pressure monitor 100′ as a result of blood flow between the inner surfaces of the needle lumen 92c and the main channel 91c and the outer surface of the guidewire 93. This sizing of the guidewire 13, 23, 33, 43, 53, 63, 73, 83, 93 that permits blood flow to the pressure monitor 100, 100′ or to another placement confirmation mechanism, such as the flash port 77, even when the guidewire 13, 23, 33, 43, 53, 63, 73, 83, 93 is fully inserted into the vessel may be utilized for any of the preferred access devices 10, 20, 30, 40, 50, 60, 70, 80, 90 described herein without significantly impacting the operation or function of the devices. Specifically, the needle 92 of the ninth preferred embodiment may be utilized and adapted for use with any of the preferred access devices 10, 20, 30, 40, 50, 60, 70, 80 described herein.

Referring to FIGS. 9G and 9H, the ninth preferred access device 90 may also be configured with an alternative needle 92′. The alternative needle 92′ of the ninth preferred embodiment has similar features in comparison to the needle 92 of the ninth preferred embodiment and the same reference numbers are utilized to identify the features of the alternative needle 92′ in comparison to the needle 92 with a prime symbol “′” utilized to distinguish the features of the alternative needle 92′. The alternative needle 92′ includes an irregular or oblong inner diameter D_{L}′ that facilitates blood flow-by past the guidewire 93 when the guidewire 93 is positioned in the needle lumen 92c′ of the needle 92′. The needle lumen 92c′ of the alternative needle 92′ of the ninth preferred embodiment includes a main channel that is configured to substantially and relatively snuggly fit the guidewire 93 and an adjacent channel that is expanded and extends away from the longitudinal axis 94. The needle lumen 92c′ preferably has a consistent cross-sectional shape between the tip 92a′ and the proximal end 92b′, but is not so limited and may have a tapering or irregular cross-section. When the guidewire 93 is positioned in the needle lumen 92c′, a crescent-shaped flow channel is preferably created between an outer surface of the guidewire 93 and the inner surface of the needle lumen 92c′ in the adjacent channel of the needle lumen 92c′ that permits blood flow through the needle lumen 92c′ past the guidewire 93. The guidewire 93 may, therefore, be loaded into the needle lumen 92c′ during insertion of the needle 92′ into the patient and blood flash is perceived when the tip 92a′ is positioned in the vessel because flash blood flows through the adjacent channel. The alternative needle 92′ is not limited to having the cross-sectional shape shown in FIGS. 9G and 9H and may have nearly any size and shape that permits flow of blood past the guidewire 93 when the guidewire 93 is positioned in the needle lumen 92c′ and that otherwise permits the needle 92′ to function as described herein.

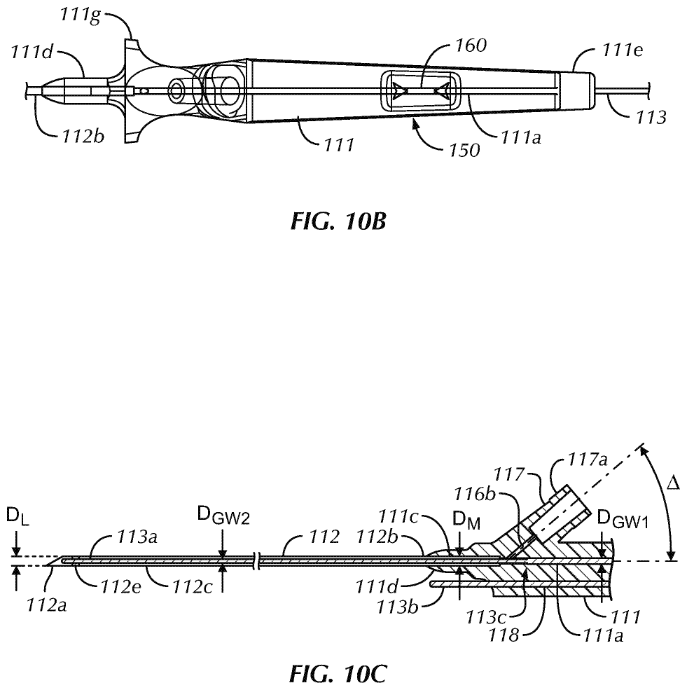

Referring to FIGS. 10A-10C, a tenth preferred access device 110 has a similar construction to the ninth preferred access device 90 and like reference numbers are utilized to identify like features of the tenth preferred access device 110 with a number “11” prefix replacing the “9” prefix to distinguish the features of the tenth preferred access device 110 from the ninth preferred access device 90.

The tenth preferred access device 110 includes an alignment window 150 on the hub 111 between the distal end 111e and the proximal end 111d and the guidewire 113 includes an indicator 160 between its proximal and distal ends, preferably on or near its front end portion 113a. The indicator 160 is preferably a visual indicator 160 and may be aligned with the alignment window 150 to ensure the guidewire 113 is positioned so that it doesn't block the flash port 117 in an initial configuration or delivered configuration. The indicator 160 is preferably positioned in the alignment window 150 in the initial configuration to ensure correct placement of the guidewire 113 within the guidewire channel 111a. The indicator 160 may be a pad printed mark, a mark applied by tampography, a laser mark, a chemically etched mark, a physical indentation on the guidewire 113 or a like mark that is located at a predetermined position on the guidewire 113 to position the guidewire 113 relative to the hub 111.

When the indicator 160 is aligned with alignment window 150, the guidewire 113 is positioned within the access device 110 such that the flash port 117 is not obstructed. The alignment window 150 and indicator 160 allow the user to confirm alignment of the guidewire 113 relative to the hub 111 and the needle 112 before use in the initial configuration and to re-position the guidewire 113 during use. The indicator 160 is not limited to being a visual indicator 160 and may be comprised of a tactile indicator or an electrically actuated indicator that provides an indication to the user when the guidewire 113 is positioned appropriately to commence the access procedure in the initial configuration, as described herein. In addition, the alignment window 150 is not limited to being comprised of a window, as shown in FIGS. 10A and 10B, and may be comprised of a visual marking on the hub 111 that may be aligned with the indicator 160 on the guidewire 113 for positioning the guidewire 113 relative to the hub 111. In addition, the initial configuration is not limited to having the front end portion 113a of the guidewire 113 positioned proximally relative to the flash port 117 to avoid blocking the flash port 117 and may be otherwise configured, such as having the distal end of the guidewire 113 positioned close, but proximate relative to the tip 112a of the needle 112, particularly in configurations where the needle 112 and guidewire 113 are configured to permit blood flow through the needle 112 even when the guidewire 113 is positioned in the needle 112, such as the configurations of the guidewire 93, the needle 92 and the alternative needle 92′ of the ninth preferred embodiment shown in FIGS. 9F-9H.

The tenth preferred access device 110 may also include an alternatively configured guidewire 113 that may be utilized with any of the preferred access devices 10, 20, 30, 40, 50, 60, 70, 80, 90, 110, 120 described herein. The alternative or tiered guidewire 113 has a proximal guidewire diameter D_{GW1} and a distal guidewire diameter D_{GW2} with a diameter transition zone 113c between the proximal and distal guidewire diameter D_{GW1}, D_{GW2}. In the tenth preferred embodiment, the diameter transition zone 113c is preferably positioned within the guidewire channel 111a proximate the intersection of the auxiliary channel 111b with the guidewire channel 111a and the main channel 111c in the initial configuration. The front end portion 113a of the guidewire 113 is also preferably positioned near, but proximally relative to the tip 112a of the needle 112 when the diameter transition zone 113c is positioned near the intersection of the auxiliary channel 111b with the guidewire channel 111a and the main channel 111c. The proximal guidewire diameter D_{GW} is preferably slightly smaller, but similar to the diameter of the guidewire channel 111a and the distal guidewire diameter D_{GW2} is preferably smaller than the proximal guidewire diameter D_{GW1} and also smaller than the inner diameter D_{L} of the needle lumen 112c of the needle 112. In the initial configuration, the guidewire 113 is positioned such that the distal guidewire diameter D_{GW2} is positioned within the needle 112, the main channel 111c and extends at least to the intersection of the main channel 111c with the auxiliary channel 111b such that blood may flow around the guidewire 113 and through the needle 112 at least to the auxiliary channel 111b. Allowing blood flow around the guidewire 113 and the through the inside of the needle lumen 112c of the needle 112 to the auxiliary channel 111b accommodates detection of the blood pressure at the flash port 117 by attaching the pressure monitor 100 or the alternative pressure monitor 100′ or by visual detection blood flow by permitting the flow out of the flash port 117. Flow or pressure is preferably detected when the tip 112a of the needle 112 is positioned within the target vessel of the patient. The tip or front end portion 113a of the guidewire 113 is also preferably positioned near the tip 112a of the needle 112, such that the guidewire 113 may be quickly urged into the vessel following positioning of the tip 112a in the vessel. The guidewire 113 is preferably configured such that the length of the guidewire 113 between the diameter transition zone 113c and the front end portion 113a of the guidewire 113 results in the diameter transition zone 113c being positioned proximally relative to the intersection of the auxiliary channel 111b and the main channel 111c when the tip 113a is positioned proximally relative to the tip 112a of the needle 112. This configuration is preferred so that a user is able to visually determine that the diameter transition zone 113c is located proximally relative to the intersection of the auxiliary channel 111b and the main channel 111c, thereby allowing blood flow into the auxiliary channel 111b through the needle 112. Alternatively, the guidewire 113 may be configured to have the visual indicator 160 that is aligned with the alignment window 150 in the initial configuration, resulting in the diameter transition zone 113c being positioned proximally relative to the intersection of the auxiliary channel 111b and the main channel 111c and the tip 113a of the guidewire 113 being positioned proximally relative to the tip 112a of the needle 112.

The tenth preferred access device 110 may also be utilized by attaching a syringe (not shown) to the flashport 97 at the attachment 97a. As the tip 92a is advanced into the patient, vacuum is drawn in the syringe, the auxiliary channel 91b, the main channel 91c and the needle lumen 92c. The sealing mechanism 95 generally isolates the guidewire channel 93a from the vacuum to maintain vacuum for drawing blood or fluid out of the patient. When the tip 92a reaches the inside of the lumen of the vessel, blood is drawn into the access device 90 by the vacuum to alert the medical professional that the tip 92a is in the vessel. This technique is preferably utilized to gain access to veins where pressure of the blood is typically lower than in an artery. The guidewire 93 may then be immediately advanced through the sealing mechanism 95, through the needle lumen 92c and into the vessel.

The tenth preferred embodiment may specifically be configured such that the distal guidewire diameter D_{GW2} is approximately eighteen thousandths of an inch (0.018″), the proximal guidewire diameter D_{GW1} is approximately thirty-five thousandths of an inch (0.035″) and the needle 112 is comprised of an eighteen gage needle with an inner diameter of approximately thirty-eight thousands of an inch (0.038″). In this preferred and non-limiting example tenth preferred embodiment, the rear end portion 113b of the guidewire 113 is positioned proximally relative to the diameter transition zone 113c and fits relatively snuggly in the guidewire channel 111a and the front end portion 113a of the guidewire 113 is positioned distally relative to the diameter transition zone 113c to provide space between the outer surface of the front end portion 113a of the guidewire 113 and the inner surface of the needle 112, at least up to a mouth of the auxiliary channel 111b in the initial configuration. The tenth preferred access device 110 is not limited to having these above-listed example preferred dimensions and may include alternative dimensions that facilitate blood flow through the needle lumen 112c of the needle 112 when the distal portion of the guidewire 113 is positioned within the needle 112 in the initial configuration. In addition, the alternative guidewire 113 is not limited to having the relatively sharp diameter transition zone 113c shown in FIG. 10C and may include a gradually sloping diameter transition zone 113c or an alternative irregular diameter transition zone 113c that results in the distal guidewire diameter D_{GW2} being smaller than the proximal guidewire diameter D_{GW1} and permitting blood flow for detection purposes, as is described herein.

The reduced distal guidewire diameter D_{GW2} of the alternative preferred guidewire 113 is also relatively atraumatic when urged into the patient's vessel. The proximal guidewire diameter D_{GW1} is also preferably configured to have a sufficient structure for advancing the guidewire 113 into the vessel and for advancing secondary instruments and components over the guidewire 113 during operation. The relatively modest distal guidewire diameter D_{GW2} is substantially atraumatic as this reduced diameter has increased flexibility and sufficient stiffness, resulting in a preferably more atraumatic guidewire 113 at the guidewire tip 113a or at least along the length of the guidewire 113 having the distal guidewire diameter D_{GW2}.

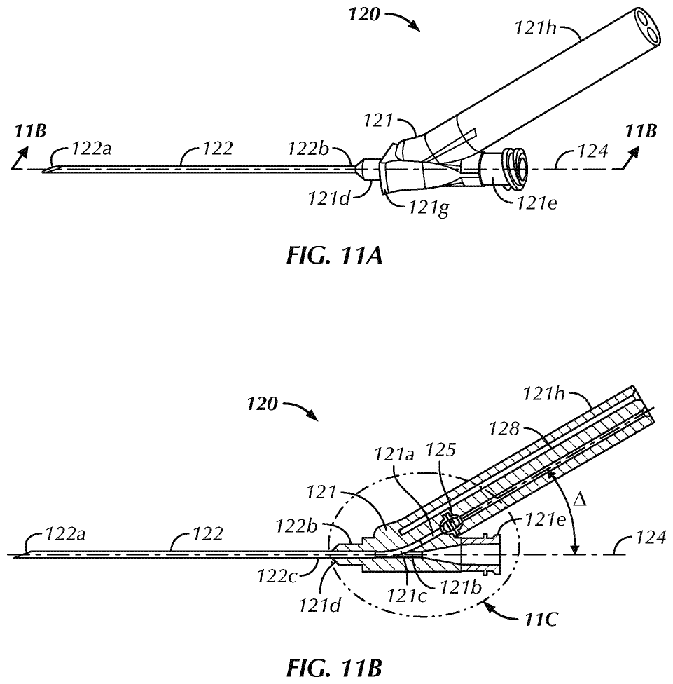

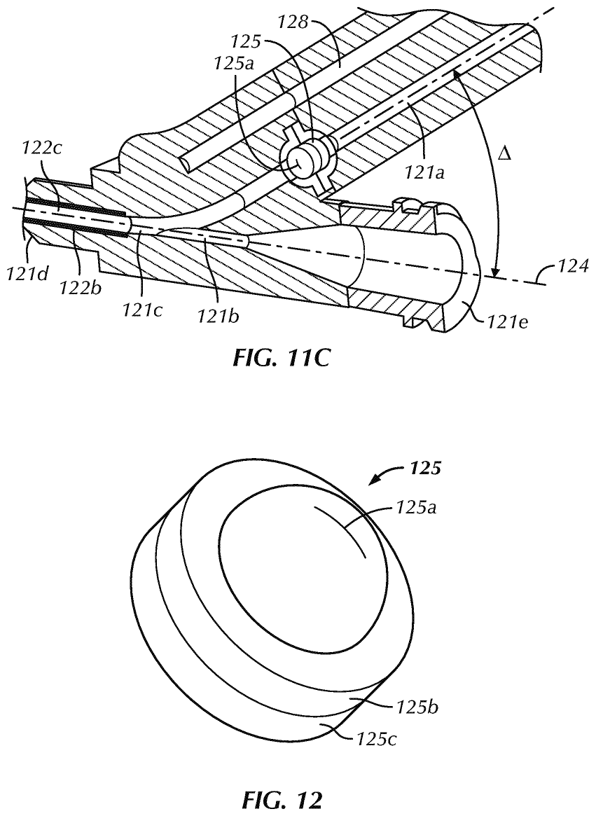

Referring to FIGS. 11A-12, an eleventh preferred access device 120 has a similar construction to the tenth preferred access device 110 and like reference numbers are utilized to identify like features of the eleventh preferred access device 120 with a number “12” prefix replacing the “11” prefix to distinguish the features of the eleventh preferred access device 120 from the tenth preferred access device 110.

The eleventh preferred access device 120 includes a Luer fitting at the proximal end 121e of the hub 121 that may be engaged by a syringe or may remain open and utilized essentially as a flashport, preferably for arterial access. The guidewire channel 121a includes the guidewire seal 125 therein, near a distal end of the guidewire channel 121a. The guidewire seal 125 preferably blocks blood flow through the guidewire channel 121a, but permits insertion of the guidewire 13 through the guidewire seal 125 when desired by the user.

When operating the eleventh preferred access device 120, the guidewire seal 125 provides a selectively permeable seal for the guidewire channel 121a. When attempting venous access, the syringe is attached to the Luer fitting at the distal end 121e of the hub 121 and vacuum is applied by the syringe to the plunger or auxiliary channel 121b, the main channel 121c and the needle lumen 122c. The vacuum is preferably applied after the needle 122 is inserted below the surface of the patient's skin. The guidewire seal 125 and positioning of the syringe on the Luer fitting prevent air from leaking into the syringe and the plunger or auxiliary channel 121b from the guidewire lumen 121a. As soon as the needle tip enters the blood vessel, the blood flashes into the syringe through the needle lumen 122c, the main channel 121c and the auxiliary channel 121b, thereby indicating a vessel strike. Then the guidewire 13 is advanced through the guidewire seal 125. The guidewire seal 125 may be comprised of multiple sealing materials and components stacked together, which have one or more precut slits 125a, or a single sealing material. The guidewire seal 125 is configured to allow the guidewire 13 to be urged therethrough, potentially through the slit 125a, into the distal end of the guidewire channel 121, the main channel 121c, the needle lumen 122c and into the blood vessel.

In the eleventh preferred embodiment, the guidewire seal 125 is constructed of a compliant material that is capable of maintaining a seal, even with the precut slit(s) 125a, but is also flexible enough to allow the guidewire 13 to pass therethrough. The eleventh preferred embodiment of the guidewire seal 125 includes two valves or valve materials 125b, 125c (sandwiched, back to back) so that (1) air is generally prevented from entering the syringe or the auxiliary channel 121b from the guidewire channel 121a when vacuum applied to the auxiliary channel 121a by the syringe and (2) blood is prevented from entering the guidewire channel 121a, at least in the proximal end proximally relative to the guidewire seal 125 due to blood pressure. The guidewire seal 125 is not limited to being comprised of the two valves or valve materials 125b, 125c stacked back-to-back and a single valve or a plurality of valve materials may be sufficient to perform the functions of the guidewire seal 125. The valve materials 125b, 125c preferably includes a dome-shaped central portion and a ring-shaped peripheral portion that is positionable into a fitting in the hub 121 at the guidewire channel 121a. The hub 121 of the eleventh preferred embodiment includes a guidewire hub attachment 121h that is removably replaceable from the hub 121 and includes portions of the guidewire channel 121a and the guidewire holder 128 therein. The guidewire seal 125 is preferably mounted to the hub 121 where the guidewire hub attachment 121h attaches to the hub 121 such that the guidewire seal 125 may be removed and replaced. The access device 120 of the eleventh preferred embodiment is not limited to including the removably guidewire hub attachment 121h and the complete hub 121 may be co-molded with the guidewire seal 125 included therein.

In the eleventh preferred embodiment, the auxiliary channel 121b with the Luer fitting at the distal end is coaxial with the longitudinal axis 124, the main channel 121c and the needle lumen 122c. The guidewire channel 121a is oriented at the acute angle Δ relative to the longitudinal axis with the guidewire holder 128 extending substantially parallel to the guidewire channel 121a in the guidewire hub attachment 121h and the hub 121. The auxiliary channel 121b, the guidewire channel 121a and the guidewire holder 128 are not limited to these preferred positions and orientations, for example the auxiliary channel 121b may be positioned at the acute angle Δ with the guidewire channel 121a and the guidewire holder 128 extending parallel or coaxial with the longitudinal axis 124 without significantly impacting the function of the preferred access device 120.

All of the preferred access devices 10, 20, 30, 40, 50, 60, 70, 80, 90, 110, 120 could contain features that provide additional guidewire management. For example, the access device 60 of the sixth preferred embodiment includes the guidewire holder 68 to hold the rear end portion 63b of the guidewire 63 in the hub 61. As another example, the access device 30 of the third preferred embodiment includes the guidewire clip 31x on the hub 30 so that the guidewire 33 can be looped to prevent the free or distal end from hanging free/loose. The guidewire tube 78b of the seventh preferred embodiment may also be utilized to manage the positioning and use of the guidewire 73.

Referring to FIGS. 1-12, the guidewire sealing mechanisms 15, 25, 35, 45, 55, 95, 125 could be designed and configured in a number of different ways, with the preferred access devices 10, 20, 30, 40, 50, 60, 70, 80, 90, 110, 120 including sealing around the guidewire 13, 23, 33, 43, 53, 63, 73, 83, 93, 113 or sealing the pathway of the guidewire 13, 23, 33, 43, 53, 63, 73, 83, 93, 113 such that the guidewire 13, 23, 33, 43, 53, 63, 73, 83, 93, 113 pierces through the seal 15, 25, 35, 45, 55, 95, 125 when advanced into the hub 11, 21, 31, 41, 51, 61, 71, 81, 91, 111, 121 after the needle 12, 22, 32, 42, 52, 62, 72, 82, 92, 112, 122 is placed in the vessel. For example, the sealing mechanism 15, 25, 35, 45, 55, 95, 125 may be positioned nearly anywhere along the length of the guidewire channel 11a, 21a, 31a, 41a, 51a, 61a, 71a, 81a, 91a, 111a, 121a and may include single or multiple portions 125b, 125c. In addition, the sealing mechanism 15, 25, 35, 45, 55, 95, 125 may include the precut slit 125a through which the guidewire 13, 23, 33, 43, 53, 63, 73, 83, 93, 113 extends to gain access to the vessel and maintain the seal to the proximal portion of the guidewire channel 121a.

All of the preferred access devices 10, 20, 30, 40, 50, 60, 70, 80, 90, 110, 120 incorporate basic ergonomic features that preferably make the access devices 10, 20, 30, 40, 50, 60, 70, 80, 90, 110, 120 intuitive to use. In addition, the preferred access devices 10, 20, 30, 40, 50, 60, 70, 80, 90, 110, 120 may be utilized with nearly any device or instrument where access to a blood vessel is desired or required. For example, the preferred access devices 10, 20, 30, 40, 50, 60, 70, 80, 90, 110, 120 may be utilized to gain access to a vessel for use with the systems of US Patent Application No. 2014/0243873, titled “Fluoroscopy-Independent Balloon Guided Occlusion Catheter and Methods;” European Patent Application No. 15806534.2, published as European Publication No. 3077036 and titled, “Conduit Guiding Tip;” International Patent Application Publication No. WO 2015/191685, titled “Conduit Guiding Tip” and US Patent Application

Publication No. 2016/0213893, titled “Low Profile Occlusion Catheter,” the contents of each of which are incorporated herein by reference in their entirety. The preferred access devices 10, 20, 30, 40, 50, 60, 70, 80, 90, 110, 120 may be utilized to gain access to an appropriate blood vessel and introduction of an introducer sheath, which is utilized to insert the systems described in the above-listed patent applications to perform procedures, such as a resuscitative endovascular balloon occlusion of the aorta (“REBOA”) procedure.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present disclosure.

We claim:

1. An access device for accessing a lumen of a blood vessel, the access device comprising:
a hub (11) having a longitudinal axis (14), a main channel (11c), a guidewire channel (11a) and an auxiliary channel (11b);
a needle (12) having a tip (12a), a proximal end (12b) and a needle lumen (12c) extending between the tip (12a) and the proximal end (12b), the proximal end (12b) secured to a distal end (11d) of the hub (11), the needle lumen (12c) being in fluid communication with the main channel (11c); and
a guidewire (13) having a rear end portion (13b) and a front end portion (13a), the front end portion (13a) of the guidewire (13) positioned in the guidewire channel (11a) in an initial configuration, the guidewire (13) positioned proximally relative to a sealing mechanism (15, 45, 95) in the guidewire channel (11a), the sealing mechanism (95) substantially limiting fluid flow from the main channel (11c) through the guidewire channel (11a).

2. The access device of claim 1, wherein the main channel (11c) and the guidewire channel (11a) are positioned coaxially on the longitudinal axis (14), the auxiliary channel (11b) extending at an acute angle (Δ) relative to the longitudinal axis (14).

3. The access device of claim 1, wherein the auxiliary channel (11b) and the main channel (11c) are positioned coaxially on the longitudinal axis (14), the guidewire channel (11a) extending at an acute angle (Δ) relative to the longitudinal axis (14).

4. The access device of claim 1, further comprising:
a plunger (16) slidably positioned in the hub (11), the hub (11) having a cylinder (11f), the cylinder (11f) being in fluid communication with the auxiliary channel (11b), the guidewire channel (11a) extending at an acute angle (Δ) relative to the longitudinal axis (14).

5. The access device of claim 1, further comprising:
a flash port (57) associated with the hub (11), the auxiliary channel (11b) extending at an acute angle (Δ) relative to the longitudinal axis (14) between the main channel (11c) and the flash port (57).

6. The access device of claim 1, further comprising: a guidewire holder (68) in the hub (11).

7. The access device of claim 6, wherein the guidewire holder (68) is open at a proximal end (11e) of the hub (11) and closed at a distal end (11d) of the hub (11), the guidewire holder (68) extending substantially parallel to the longitudinal axis (14).

8. The access device of claim 6, wherein the rear end portion (13b) of the guidewire (13) is positioned in the guidewire holder (68) in the initial configuration.

9. The access device of claim 1, further comprising:
a pressure monitor (100) connected to the hub (11) to monitor fluid pressure in the main channel (11c) and the auxiliary channel (11b).

10. The access device of claim 9, wherein the pressure monitor (100) includes a display (100a).

11. The access device of claim 9, wherein the pressure monitor (100) is selected from the group consisting of an external pressure monitor, a built-in pressure monitor, a mechanical gauge, an electronic pressure monitor, a go or no-go indicator and an Arterial vs Vein Indication Device.

12. The access device of claim 9, wherein the pressure monitor (100) includes an electronic pressure transducer.

13. The access device of claim 1, wherein the guidewire (13) has an outside diameter (DGW), the main channel (11c) has an inside diameter (DM) and the needle lumen (12c) has an inside diameter (DL), the outside diameter (DGW) being smaller than the inside diameters (D_{M}, D_{L}) of the needle lumen (12c) and the main channel (11c) such that fluid is able to flow past the guidewire (13) in the needle lumen (12c) and the main channel (11c) between an outer surface of the guidewire (13) and inner surfaces of the needle lumen (12c) and the main channel (11c).

14. An access device for accessing a lumen of a blood vessel, the access device comprising:
a hub (11) having a longitudinal axis (14), a main channel (11c), a guidewire channel (11a), a guidewire holder (68) and an auxiliary channel (11b), the main channel (11c) and the guidewire channel (11a) extending substantially parallel to the longitudinal axis (14), the auxiliary channel (11b) extending at an acute angle (Δ) relative to the longitudinal axis (14);
a needle (12) having a tip (12a), a proximal end (12b) and a needle lumen (12c) extending between the tip (12a) and the proximal end (12b), the proximal end (12b) secured to a distal end (11d) of the hub (11), the needle lumen (12c) being in fluid communication with the main channel (11c) and extending substantially parallel to the longitudinal axis (14); and
a guidewire (13) having a rear end portion (13b) and a front end portion (13a), the front end portion (13a) of the guidewire (13) positioned in the guidewire channel (11a) in an initial configuration and the rear end portion (13b) positioned in the guidewire holder (68) in the initial configuration, the front end portion (13a) of the guidewire (13) positioned proximally relative to a sealing mechanism (15, 45, 95) in the guidewire channel (11a), the sealing mechanism (95) positioned proximate the main channel (11c) to substantially limit fluid flow from the main channel (11c) through the guidewire channel (11a).

15. The access device of claim 14, wherein a distal end of the auxiliary channel (11b) includes an attachment for releasable engagement of a pressure monitor (100).

16. The access device of claim 15, wherein the attachment is selected from the group consisting of a Luer-Lock style connector, a bayonet-style connector and mechanical threads.

17. The access device of claim 14, wherein the guidewire holder extends substantially parallel to the longitudinal axis (14) from a distal end (91d) to a proximal end (91e) of the hub (11).

18. The access device of claim 14, wherein the hub (11) includes finger grooves (91g) on a distal end (11d, 91d) of the hub (11).

19. The access device of claim 14, further comprising:
a pressure monitor (100) selectively mountable to the hub (11), the pressure monitor being in fluid communication with the auxiliary channel (11b) when mounted to the hub (11).

20. The access device of claim 19, wherein the pressure monitor (100) is selected from the group consisting of an external pressure monitor, a mechanical gauge, an electronic pressure monitor, a go or no-go indicator and an Arterial vs Vein Indication Device.
